SEQUENCE LISTING (85 sequences)

SEQ ID NO: 1 - protein, 9 aa, Homo Sapiens
SLLMWITQC

SEQ ID NO: 2 - protein, 9 aa, Artificial Sequence
  Peptide analogue
SLLMWITQV

SEQ ID NO: 3 - protein, 9 aa, Artificial Sequence
  Peptide analogue
SXLMWITQV

SEQ ID NO: 4 - protein, 9 aa, Artificial Sequence
  Peptide analogue
SILMWITQV

SEQ ID NO: 5 - protein, 9 aa, Artificial Sequence
  Peptide analogue
SVLMWITQC

SEQ ID NO: 6 - protein, 9 aa, Artificial Sequence
  Peptide analogue
FLLMWITQV

SEQ ID NO: 7 - protein, 9 aa, Artificial Sequence
  Peptide analogue
FILMWITQV

SEQ ID NO: 8 - protein, 9 aa, Artificial Sequence
  Peptide analogue
FVLMWITQV

SEQ ID NO: 9 - protein, 9 aa, Artificial Sequence
  Peptide analogue
KLLMWITQV

SEQ ID NO: 10 - protein, 9 aa, Artificial Sequence
  Peptide analogue
KVLMWITQV

SEQ ID NO: 11 - protein, 9 aa, Artificial Sequence
  Peptide analogue
KXLMWITQV

SEQ ID NO: 12 - protein, 9 aa, Artificial Sequence
  Peptide analogue
TVLMWITQV

SEQ ID NO: 13 - protein, 9 aa, Artificial Sequence
  Peptide analogue
YLLMWITQV

SEQ ID NO: 14 - protein, 10 aa, Artificial Sequence
  Peptide analogue
YLLMWITQVX

SEQ ID NO: 15 - protein, 9 aa, Artificial Sequence
  Peptide analogue
YLLMWATQV

SEQ ID NO: 16 - protein, 9 aa, Artificial Sequence
  Peptide analogue
YLLMWLTQV

SEQ ID NO: 17 - protein, 9 aa, Artificial Sequence
  Peptide analogue
YLLMWNTQV

SEQ ID NO: 18 - protein, 9 aa, Artificial Sequence
  Peptide analogue
YLLMWTTQV

SEQ ID NO: 19 - protein, 9 aa, Artificial Sequence
  Peptide analogue
YXLMWITQV

SEQ ID NO: 20 - protein, 9 aa, Artificial Sequence
  Peptide analogue
YILMWITQV

SEQ ID NO: 21 - protein, 9 aa, Artificial Sequence
  Peptide analogue
YVLMWITQV

SEQ ID NO: 22 - protein, 9 aa, Artificial Sequence
  Peptide analogue
YLLAWITQV

SEQ ID NO: 23 - protein, 9 aa, Artificial Sequence
  Peptide analogue
YLLIWITQV

SEQ ID NO: 24 - protein, 9 aa, Artificial Sequence
  Peptide analogue
YLLLWITQV

SEQ ID NO: 25 - protein, 9 aa, Artificial Sequence
  Peptide analogue
YLLNWITQV

SEQ ID NO: 26 - protein, 9 aa, Artificial Sequence
  Peptide analogue
YLLVWITQV

SEQ ID NO: 27 - protein, 9 aa, Artificial Sequence
  Peptide analogue
YLLMWITAV

SEQ ID NO: 28 - protein, 9 aa, Artificial Sequence
  Peptide analogue
YLLMWITEV

SEQ ID NO: 29 - protein, 9 aa, Artificial Sequence
  Peptide analogue
YLLMWITNV

SEQ ID NO: 30 - protein, 9 aa, Artificial Sequence
  Peptide analogue
YLLMWITQC

SEQ ID NO: 31 - protein, 9 aa, Artificial Sequence
  Peptide analogue
FLLMWITQC

SEQ ID NO: 32 - protein, 9 aa, Artificial Sequence
  Peptide analogue
KLLMWITQC

SEQ ID NO: 33 - protein, 9 aa, Artificial Sequence
  Peptide analogue
FVLMWITQC

SEQ ID NO: 34 - protein, 9 aa, Artificial Sequence
  Peptide analogue
FILMWITQC

SEQ ID NO: 35 - protein, 9 aa, Artificial Sequence
  Peptide analogue
YVLMWITQC

SEQ ID NO: 36 - protein, 9 aa, Artificial Sequence
  Peptide analogue
YILMWITQC

SEQ ID NO: 37 - protein, 9 aa, Artificial Sequence
  Peptide analogue
YLLMWITQL

SEQ ID NO: 38 - protein, 9 aa, Artificial Sequence
  Peptide analogue
YLLMWITQA

SEQ ID NO: 39 - protein, 9 aa, Artificial Sequence
  Peptide analogue
YLLMWITQI

SEQ ID NO: 40 - protein, 9 aa, Artificial Sequence
  Peptide analogue
FLLMWITQL

SEQ ID NO: 41 - protein, 9 aa, Artificial Sequence
  Peptide analogue
FLLMWITQA

SEQ ID NO: 42 - protein, 9 aa, Artificial Sequence
  Peptide analogue
FLLMWITQI

SEQ ID NO: 43 - protein, 9 aa, Artificial Sequence
  Peptide analogue
KLLMWITQL

SEQ ID NO: 44 - protein, 9 aa, Artificial Sequence
  Peptide analogue
KLLMWITQA

SEQ ID NO: 45 - protein, 9 aa, Artificial Sequence
  Peptide analogue
KLLMWITQI

SEQ ID NO: 46 - protein, 9 aa, Artificial Sequence
  Peptide analogue
YLLMWITQX

SEQ ID NO: 47 - protein, 9 aa, Artificial Sequence
  Peptide analogue
YLLMWITQX

SEQ ID NO: 48 - protein, 9 aa, Artificial Sequence
  Peptide analogue
YLLMWITQX

SEQ ID NO: 49 - protein, 9 aa, Artificial Sequence
  Peptide analogue
YVLMWITQL

SEQ ID NO: 50 - protein, 9 aa, Artificial Sequence
  Peptide analogue
YVLMWITQA

SEQ ID NO: 51 - protein, 9 aa, Artificial Sequence
  Peptide analogue
YVLMWITQI

SEQ ID NO: 52 - protein, 9 aa, Artificial Sequence
  Peptide analogue
YILMWITQL

SEQ ID NO: 53 - protein, 9 aa, Artificial Sequence
  Peptide analogue
YILMWITQA

SEQ ID NO: 54 - protein, 9 aa, Artificial Sequence
  Peptide analogue
YILMWITQI

SEQ ID NO: 55 - protein, 9 aa, Artificial Sequence
  Peptide analogue
FVLMWITQL

SEQ ID NO: 56 - protein, 9 aa, Artificial Sequence
  Peptide analogue
FVLMWITQA

SEQ ID NO: 57 - protein, 9 aa, Artificial Sequence
  Peptide analogue
FVLMWITQI

SEQ ID NO: 58 - protein, 9 aa, Artificial Sequence
  Peptide analogue
FILMWITQL

SEQ ID NO: 59 - protein, 9 aa, Artificial Sequence
  Peptide analogue
FILMWITQA

SEQ ID NO: 60 - protein, 9 aa, Artificial Sequence
  Peptide analogue
FILMWITQI

SEQ ID NO: 61 - protein, 9 aa, Artificial Sequence
  Peptide analogue
YLLMWVTQV

SEQ ID NO: 62 - protein, 9 aa, Artificial Sequence
  Peptide analogue
YLLMWITDV

SEQ ID NO: 63 - protein, 9 aa, Artificial Sequence
  Peptide analogue
YLLMWITTV

SEQ ID NO: 64 - protein, 9 aa, Artificial Sequence
  Peptide analogue
YLLMWITSV

SEQ ID NO: 65 - protein, 10 aa, Artificial Sequence
  Peptide analogue
YLLMWITQLX

SEQ ID NO: 66 - protein, 10 aa, Artificial Sequence
  Peptide analogue
YLLMWITQAX

SEQ ID NO: 67 - protein, 9 aa, Artificial Sequence
  Peptide analogue
SLLMWITQL

SEQ ID NO: 68 - protein, 9 aa, Artificial Sequence
  Peptide analogue
SLLMWITQA

SEQ ID NO: 69 - protein, 9 aa, Artificial Sequence
  Peptide analogue
SLLMWITQI

SEQ ID NO: 70 - protein, 10 aa, Homo Sapiens
SLLMWITQCF

SEQ ID NO: 71 - protein, 10 aa, Artificial Sequence
  Peptide analogue
SLLMWITQCL

SEQ ID NO: 72 - protein, 10 aa, Artificial Sequence
  Peptide analogue
YLLMWITQVL

SEQ ID NO: 73 - protein, 10 aa, Artificial Sequence
  Peptide analogue
YLLMWITQVI

SEQ ID NO: 74 - protein, 10 aa, Artificial Sequence
  Peptide analogue
YLLMWITQVV

SEQ ID NO: 75 - protein, 10 aa, Artificial Sequence
  Peptide analogue
YLLMWITQVX

SEQ ID NO: 76 - protein, 10 aa, Artificial Sequence
  Peptide analogue
YLLMWITQVX

SEQ ID NO: 77 - protein, 9 aa, Artificial Sequence
  Peptide analogue
SVLMWITQL

SEQ ID NO: 78 - protein, 9 aa, Artificial Sequence
  Peptide analogue
SVLMWITQI

SEQ ID NO: 79 - protein, 9 aa, Artificial Sequence
  Peptide analogue
SVLMWITQV

SEQ ID NO: 80 - protein, 9 aa, Artificial Sequence
  Peptide analogue
SILMWITQL

SEQ ID NO: 81 - protein, 9 aa, Artificial Sequence
  Peptide analogue
YXLMWITQV

SEQ ID NO: 82 - protein, 9 aa, Artificial Sequence
  Peptide analogue
WVLMWITQV

SEQ ID NO: 83 - protein, 9 aa, Artificial Sequence
  Peptide analogue
SVLMWITQA

SEQ ID NO: 84 - protein, 9 aa, Artificial Sequence
  Peptide analogue
SILMWITQC

SEQ ID NO: 85 - protein, 9 aa, Artificial Sequence
  Peptide analogue
SILMWITQA

(19) United States
(12) Patent Application Publication
(10) Pub. No.: US 2013/0058965 A1
(43) Pub. Date: Mar. 7, 2013

(54) INJECTABLE FORMULATIONS FOR PARENTERAL ADMINISTRATION

(71) Applicants: Jim Ferguson, Austin, TX (US); Liping Liu, Manassas, VA (US)

(72) Inventors: Jim Ferguson, Austin, TX (US); Liping Liu, Manassas, VA (US)

(73) Assignee: MANNKIND CORPORATION, Valencia, CA (US)

(21) Appl. No.: 13/520,160

(22) PCT Filed: Dec. 30, 2010
(86) PCT No.: PCT/US2010/062612

§ 371 Date: Nov. 8, 2012

Related U.S. Application Data
(60) Provisional application No. 61/291,834, filed on Dec. 31, 2009.

(51) Int. Cl. A61K 39/00; A61P 37/04; A61P 35/00

(52) U.S. Cl. 424/185.1

(57) ABSTRACT

Disclosed herein are uses, methods, and processes for preparing or manufacturing a lyophilized cake that comprises a water-insoluble agent, wherein the cake is capable of being disintegrated in a parenterally acceptable solvent to form a syringeable liquid suspension of fine particles of the water-insoluble active agent that is suitable for pharmaceutical uses. Lyophilized cakes prepared according to the methods disclosed herein and kits containing such lyophilized cakes are also disclosed. Further disclosed herein are methods and processes for preparing a syringeable liquid suspension of fine particles of the water-insoluble active agent that is suitable for pharmaceutical uses.

### FIELD OF THE DISCLOSURE

The disclosure relates generally to pharmaceutical formulations. More particularly, disclosed herein are methods and processes for preparing injectable formulations for use as, or with, therapeutic or prophylactic compositions comprising immunogenic compositions including, for example, compositions capable of producing a desired immune responses in subjects to whom the compositions are administered. Also described herein are methods and processes for preparing a syringeable liquid suspension of fine particles of a water-insoluble active agent that is suitable for pharmaceutical uses.

### BACKGROUND

The success and therapeutic effectiveness of a pharmaceutical formulation or composition depends not only on its efficacy in treating a disease or condition, but also upon pharmaceutical properties such as stability, solubility, sterility, and an efficacious dosage/amount to obtain the desired immunogenic, prophylactic or therapeutic response. Producing pharmaceutical formulations or compositions that possess the aforementioned pharmaceutical properties while still fulfilling the requirements of being non-toxic, economical to manufacture, formed of readily available components, and being consistent with respect to final composition and physical characteristics, including, solubility and stability, is an ongoing challenge in the art.

One approach to addressing these challenges lies in the methodology and processes used to prepare or manufacture such pharmaceutical formulations or compositions.

### SUMMARY

The following embodiments and aspects thereof are described and illustrated in conjunction with formulations, compositions, methods and processes which are meant to be exemplary and illustrative, not necessarily limiting in scope.

Embodiments of the invention include methods for preparing a lyophilized cake including a water-insoluble agent, wherein the lyophilized cake is capable of being disintegrated in a parenterally acceptable solvent to form an injectable formulation. The method can include: a) preparing a pre-lyophilization solution including: (i) one or more active agents, wherein at least one of the one or more active agents is water-insoluble; (ii) a parenterally unacceptable volatile lyophilizable solvent; and (iii) a water soluble bulking agent; b) sterile filtering the pre-lyophilization solution thereby forming a sterile solution; and c) lyophilizing the sterile solution to produce the lyophilized cake including a water-insoluble agent, wherein the lyophilized cake is capable of being disintegrated in a parenterally acceptable solvent to form an injectable formulation including a suspension of fine particles of the water-insoluble active agent. In some embodiments, the parenterally unacceptable volatile lyophilizable solvent can include a mixture of solvents.

In some embodiments, the pre-lyophilization solution can further include, for example, a surfactant. Preparing the pre-lyophilization solution can involve directly dissolving the at least one water-insoluble active agent and the water-soluble bulking agent in the parenterally unacceptable volatile lyophilizable solvent. Likewise, preparing the pre lyophilization solution can involve: a) dissolving the at least one water-insoluble active agent in the parenterally unacceptable volatile lyophilizable solvent, thereby forming a first solution; b) dissolving the water-soluble bulking agent, and optionally, the surfactant, in an aqueous solution thereby forming a second solution; c) separately sterile filtering each of the first and second solutions to form a first sterile solution and a second sterile solution, respectively; and d) adding the second sterile solution to the first sterile solution by controlled mixing to prevent aggregation or precipitation of the water-insoluble agent.

In various embodiments the water-insoluble agent can be hydrophobic. In some embodiments, the water-insoluble agent can be, for example, a small molecule, protein, peptide, or the like. In some embodiments, the water-insoluble agent can be, for example, an immunogenic, therapeutic, or prophylactic molecule, or the like. In some embodiments, the water-insoluble agent can be, for example, selected from among tumor associated antigens, tumor specific antigens, differentiation antigens, embryonic antigens, cancer-testis antigens, unique tumor antigens resulting from chromosomal translocations, viral antigens, antigens of oncogenes, mutated tumor-suppressor genes, immunogenic fragments thereof, and the like.

In some embodiments, the water-insoluble agent can be a tumor antigen selected from the group consisting of NY-ESO-1, SSX-2, Melan-A, tyrosinase, PRAME, PSMA, immunogenic fragments thereof, and the like. Likewise, in some embodiments, the immunogenic fragment can be an NY-ESO-1 immunogenic peptide, such as, for example, NY-ESO-1157-165, or an analogue thereof, or the like. The NY-ESO-1157-165 analogue can be, for example, SNvaLMWITQV (SEQ ID NO:3).

In some embodiments, the parenterally unacceptable volatile lyophilizable solvent can be an acid or base, such as, for example, acetic acid or hydrochloric acid, or the like. In some embodiments, the water-soluble bulking agent can include, for example, mannitol. In some embodiments, methods can further include a step for improving long-term stability against oxidative degradation of a hydrophobic agent having poor solubility in aqueous media, including storing the lyophilized cake under an inert gas.

Some embodiments provide methods for preparing an injectable formulation including a suspension of fine particles of a water-insoluble active agent, wherein the method can include: obtaining a sterile lyophilized cake prepared according the method of any of the embodiments herein; and disintegrating the lyophilized cake in a parenterally acceptable solvent to form a liquid fine particle suspension for administration.

Some embodiments provide methods for administering a water-insoluble active agent in a parenterally acceptable solvent, wherein the method can include: obtaining a sterile lyophilized cake prepared according the methods of any of the embodiments herein; disintegrating the lyophilized cake in a parenterally acceptable solvent to form a syringeable liquid fine particle suspension; and administering the reconstituted suspension to a patient.

In some embodiments, the suspension can be stable for about 360 minutes at room temperature. In some embodiments, the administration can be within about 360 minutes from disintegrating. In some embodiments, the administration can be directly to the lymphatic system such as, for example, administration that includes intranodal administration. In some embodiments, the lyophilized cake can be disintegrated in water suitable for injection, or in a sodium chloride solution, or in a phosphate buffer, for example. In preferred embodiments, the cake can be disintegrated in a sterile solvent and/or solution.

Embodiments of the invention also include a lyophilized cake including a water-insoluble agent, wherein the lyophilized cake is capable of being disintegrated in a parenterally acceptable solvent to form an injectable formulation including a suspension of fine particles of the water-insoluble active agent. The lyophilized cake can be prepared according to the method of any of the embodiments herein. Some embodiments include methods wherein the lyophilized cake is provided, and the parenterally acceptable solvent is added thereto, to disintegrate it and form a fine particle suspension of the water insoluble agent for administration.

Some embodiments provide a kit that can include: i) a lyophilized cake including a water-insoluble agent, wherein the lyophilized cake is capable of being disintegrated in a parenterally acceptable solvent to form an injectable formulation including a suspension of fine particles of the water-insoluble active agent, ii) a parenterally acceptable solvent for preparing a fine particle suspension prior to administration, and iii) instructions for preparing the fine particle suspension. In some embodiments of the kit, the lyophilized cake can be prepared according to the method of any of the embodiments herein. The parenterally acceptable solvent can be water for injection, a sodium chloride solution, or a phosphate buffer, for example. In preferred embodiments, the parenterally acceptable solvent can be a sterile solvent and/or solution. The kit can further include, for example, a syringe, an ampule, a vial, or the like. The syringe can include, for example, an ultrasonically opaque needle.

Some embodiments of the invention provide a pharmaceutical composition including a lyophilized cake including one or more active agents, dispersed within the cake, wherein at least one of the one or more active agents is water-insoluble, and wherein upon disintegration of the cake with a parenterally acceptable solvent, a syringeable suspension of fine particles of the water-insoluble active agent can be obtained.

### BRIEF DESCRIPTION OF THE DRAWINGS

FIGS. 1A-1C show various exemplary NY-ESO-1₁₅₇₋₁₆₅ peptide analogues for use in the lyophilized formulations described herein.

FIG. 2 is a graph illustrating the purity of a lyophilized formulation of NY-ESO at 5° C., 25° C. and 40° C. over a period of time.

FIG. 3 is a graph illustrating the percent label claim of a lyophilized formulation of NY-ESO-1 at 5° C., 25° C. and 40° C. over a period of time.

FIG. 4 is a graph showing the median (X₅₀) particle size of a lyophilized cake formulation of NY-ESO-1 at 5° C., 25° C., and 40° C. over a period of time.

FIG. 5 is a bar graph showing an evaluation of NY-ESO-1 formulations by ELISPOT analysis. MN-gamma ELISPOT analysis performed in triplicate, values (Average elispots/4X10E5) represent average+/−SEM from individual animals.

FIG. 6 is a bar graph showing an evaluation of NY-ESO-1 formulation by ⁵¹Cr-release assay. T2 cells pulsed with NY-ESO-1₁₅₇₋₁₆₅ (T2+N₁₅₇) peptide were targeted by CTLs isolated from immunized HHD-1 mice. Specific lysis values were compared to un-pulsed T2 control cells. The graph shows the result of an assay in which the percent CTL cells specific lysis of T2 cells was measured.

### DETAILED DESCRIPTION

### Definitions

Unless otherwise clear from the context of the use of a term herein, the following listed terms shall generally have the indicated meanings for purposes of this description.

As used herein “active agent” refers to a molecule, compound, or substance having or capable of having biological activity. In some embodiments, an active agent can be a molecule or compound having therapeutic or prophylactic activity, including, for example, immunogenic activity and/or diagnostic activity. An active agent can be a drug, a pharmaceutical composition or substance, a bioactive agent, and the like. An active agent can be a natural compound, an isomer, an analogue, or a derivative thereof. An active agent can be, for example, organic macromolecules including a nucleic acid, a synthetic organic compound, a protein, a polypeptide, a peptide, and the like.

As used herein, an “analogue” or “sequence analogue” can include a variant of a peptide in which one or more amino acid residues are added, deleted, inserted, modified or substituted, while still essentially maintaining the function of interest of the peptide. An amino acid residue can be added or deleted from either end of the peptide, deleted from within the peptide, inserted within the peptide, modified at one or more residues, or substituted for one or more of the residues within the peptide. Peptides, proteins, and polypeptides are all chains of amino acids linked by peptide bonds and are included in this definition.

The term “stability” or “stable,” is used herein to indicate that the physico chemical condition of a parenteral formulation has not undergone substantial chemical or physical change to render the formulation unsuitable for its intended use (e.g., loss of efficacy, inducing unacceptable side effects, or the like, or a combination thereof). Such chemical or physical changes include, for example, decomposition, breakdown, inactivation, aggregation, or agglomeration, or a combination thereof. Physical stability refers to the lack of substantial particle aggregation, and/or the uniformity of the particles substantially throughout the suspension, upon disintegration. Chemical stability refers to the lack of substantial degradation or decomposition, which can, for example, occur due to oxidation, allowing for a stable formulation to be obtained. Sufficient chemical stability is normally defined in the art as less than 5-10% degradation of the active agent composition over a 2-year time period under the specified storage conditions. In some instances, the stability can be “long term” or “short term” as used to refer to the length or period of time that a composition maintains the desired physico-chemical characteristic(s).

As used herein, a “solution” refers to a homogeneous mixture of one or more substances, components, or compositions in an aqueous or liquid media. A solution can also be a mixture of two or more solutions containing different or similar components, substances, or compositions to form a single combined solution.

As used herein, a “suspension” refers to finely dispersed particles as obtained upon disintegration of a lyophilized cake or product in a disintegration buffer or liquid that is mixed with but undissolved in said buffer or liquid. The buffer or other liquid can be a solution with respect to its other components.

As used herein, the terms “disintegrate” or “disintegration” or “disintegrated” refer to the process of dissolving the soluble components of a lyophilized cake and releasing the insoluble components from the cake into the liquid (e.g., a sterile, parenterally acceptable solvent).

The term “water-insoluble” as used herein refers to an active agent/compound that is hydrophobic or is not readily soluble (i.e., having low, poor or minimal or no solubility) in water. In some embodiments, an active agent having low solubility in water is an agent having a water solubility that is less than 0.5 mg/ml at or near neutral pH of 5 to 8 at or near room temperature; an active agent having poor solubility in water is an agent having a water solubility that is less than 5 μg/ml at or near neutral pH of 5 to 8 at or near room temperature; and an active agent having minimal solubility in water is an agent having a water solubility of less than 0.05 μg/ml at or near neutral pH of 5 to 8 at or near room temperature. In some embodiments, an active agent having low solubility in water is an agent having a water solubility that is less than 0.5 micro mol/ml at or near neutral pH of 5 to 8 at or near room temperature; an active agent having poor solubility in water is an agent having a water solubility that is less than 5 nano mol/ml at or near neutral pH of 5 to 8 at or near room temperature; and an active agent having minimal solubility in water is an agent having a water solubility of less than 0.05 nano mol/ml at or near neutral pH of 5 to 8 at or near room temperature. In some embodiments, an active agent having low solubility in water is an agent having a water solubility that is not able to form an injectable solution/formulation at a concentration that is sufficient to deliver an effective amount of the active agent to induce the desired result (e.g., a CTL response, a detectable result for diagnostic purposes, or the like). Such a concentration is referred to as an effective concentration for the purpose se of simplicity and convenience. Merely by way of example, if an active agent that can induce a CTL response has low solubility, in order to deliver an effective amount of the active agent to a subject, a volume of the formulation containing the active agent that is physiologically unacceptable has to be injected or otherwise administered to the subject. An active agent having poor solubility in water is an agent having a water solubility that is one to two orders of magnitude below the effective concentration at or near neutral pH of 5 to 8 at or near room temperature; and an active agent having minimal solubility in water is an agent having a water solubility of two to three orders of magnitude below the effective concentration at or near neutral pH of 5 to 8 at or near room temperature. The term “lipid-insoluble” as used herein refers to an active agent/compound or composition that is poorly soluble (i.e., having low or minimal or no solubility) in lipids.

The term “volatile lyophilizable solvent” and such similar terms refer to a solvent that has characteristics of being both volatile and lyophilizable. Such a solvent is volatile in that it has the characteristic of evaporating/vaporizing within a short period of time at ambient temperatures (e.g., room temperature). In addition, such a parenterally unacceptable solvent is lyophilizable in that it has the characteristic of remaining frozen in freeze drying conditions, for example, the ability to freeze dry in a range of 0 to −50 degrees Celsius, or at least 25 to −50 degrees Celsius; and is sufficiently volatile to be sublimated in a lyophilization cycle at 50 to 250 millitorrs (mmHg).

As used herein, the term “parenterally unacceptable solvent” refers to a solvent that is unacceptable in that it is toxic, carcinogenic, caustic and/or likely to cause tissue damage, allergenic, or that causes some other undesirable reaction, and thus, is not practical for administration to a subject, at the concentration employed to dissolve the agent of interest (e.g., a water-insoluble active agent disclosed herein).

As used herein, the term “lyophilized cake” or “lyophilized product” refers to a solid/cake composition, remaining after lyophilization. A lyophilized cake or lyophilized product can have moisture content generally below 10% by weight (% w) water, usually below 5% by weight and in some embodiments, less than 3% by weight.

The term “effective amount,” as used herein is the amount required to provide a desired response in the patient or subject to be treated. The precise dosage will vary according to a variety of factors, including, but not limited to, the age and size of the subject, and the disease and the treatment being effected. The “effective amount” will also be determined based on the anticipated pharmacodynamic response and/or bioavailability of the active agent(s) and/or pharmaceutical product used.

As used herein, the term “bioavailability” refers to amount of the active agent that becomes available or accessible to the target tissue or organ after administration to the subject.

As used herein, the term “syringeable” refers to the ability of a suspension of fine particles to be drawn into a syringe through a needle of an appropriate gauge and injected into a subject. Medical uses most typically involve 15 to 32 gauge needles. Some but not all embodiments are limited to this range, any range within this range, or to sizes greater than or equal to any gauge in this range. The term “injectable” can be used interchangeably with “syringeable.” “Injectable” can also further indicate that the formulation is parenterally acceptable, or physiologically acceptable, or pharmaceutically acceptable. “Injectable” can further be used to refer to a formulation that upon disintegrating is capable of being drawn up into a syringe through a needle of appropriate gauge and injected into a subject. As such, “injectable” refers to characteristics of the material whether disintegrated or not. “Syringeable” or “injectable” can further refer to a material that upon disintegration/reconstitution by conventional means results in a final product that free from anything unsuitable for injection.

As used herein, a subject can include an animal, e.g., a mammal, a human patient, or the like.

In some embodiments, the numbers expressing quantities of ingredients, properties such as molecular weight, reaction conditions, and so forth, used to describe and claim certain embodiments of the application are to be understood as being modified in some instances by the term “about.” Accordingly, at some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Embodiments of the disclosure relate to methods of preparing an injectable formulation comprising: providing one or more active agents wherein at least one of the one or more active agent is water-insoluble; dissolving the one or more active agents in a parenterally unacceptable volatile lyophilizable solvent; providing a solution comprising a water-soluble bulking (caking) agent; separately sterile filtering each of the resultant solutions containing the one or more agents and the solution containing the bulking agent; combining the sterile solutions by controlled (e.g., slow or gentle) mixing to prevent aggregation and/or precipitation of the water-insoluble agent; and lyophilizing the sterile combined solution whereby the solvent is substantially removed to produce a lyophilized cake.

In some embodiments, in a non-limiting manner, the parenterally unacceptable lyophilizable volatile solvent is a weak acid or base. In some embodiments, the volatile solvent is a strong acid or base. In some embodiments, in a non-limiting manner, the solvent is a strong polar aprotic or prude solvent. In some embodiments, the parenterally unacceptable lyophilizable volatile solvent is one solvent selected from the group consisting of acetic acid, trifluoroacetic acid (TFA), hydrochloric acid, ammonium hydroxide or solutions thereof, but is not necessarily limited to such. In some embodiments, an aprotic solvent, such as DMSO (dimethylsulfoxide) or DMF (dimethylformamide), is utilized as a co-solvent to promote dissolution of the active agent. In some embodiments, the parenterally unacceptable solvent is a solvent with sufficient volatility as to be lyophilizable.

Some embodiments of the disclosure relate to one or more active agents, wherein at least one of the one or more active agents is water-insoluble. In some embodiments, the water-insoluble agent is a hydrophobic agent or an agent having low, minimal or poor solubility in water. In some embodiments, the water-insoluble agent is a small molecule, protein, polypeptide or peptide. In some embodiments, the one or more active agent is water-insoluble and lipid-insoluble. In some embodiments, the active agent is a single agent. In some embodiments, the one or more water-insoluble agent is combined with at least one or more water-soluble agents. In some embodiments, the water-soluble agent is a protein, polypeptide, peptide or a nucleic acid encoding a peptide or polypeptide.

Some embodiments of the disclosure relate to a lyophilized product or cake. A lyophilized product and a lyophilized cake are used interchangeably, and are sometimes referred to as a cake for simplicity and convenience. In some embodiments, the cake is disintegrated in a parenterally acceptable liquid/solvent to form an injectable/syringeable formulation comprising a suspension of fine particles of one or more active agents including at least one water-insoluble active agent. In some embodiments, the parenterally acceptable liquid/solvent is water for injection, a sodium chloride solution, or a phosphate buffer, but is not necessarily limited to such. In some embodiments, the parenterally acceptable liquid/solvent further includes a surfactant. In some embodiments, the lyophilized cake is capable of being disintegrated in a parenterally acceptable liquid/solvent to form an injectable/syringeable liquid fine particle suspension for administration to a patient or subject. In some embodiments, the liquid fine particle suspension is stable from the time of suspension to 0.5 hours, or 1 hour, or 4 hours, or 6 hours, or 8 hours, or 12 hours, or 18 hours, or 24 hours, or longer than 24 hours, at room temperature. In some embodiment, the liquid fine particle suspension is stable from the time of suspension up to 6 hours at room temperature. In some embodiments, the lyophilized product or cake is disintegrated to form a suspension of a desired or target concentration of the one or more active agents based on a specific/intended application or need thereof. In some embodiments, a desired or target concentration is 0.1 mg/ml, or 0.5 mg/ml, or 1 mg/ml, or 2 mg/ml, or 5 mg/ml, or 10 mg/ml, or 20 mg/ml, or 30 mg/ml but is not necessarily limited to such. In an exemplary embodiment, the desired or target concentration is at or 1 mg/ml.

In some embodiments, at least one of the one or more active agents includes a small molecule, protein, polypeptide or peptide. In some embodiments, at least one of the one or more active agents is an immunogenic, therapeutic or prophylactic molecule. In some embodiments, at least one of the one or more active agent is selected from the group consisting of tumor associated antigens, tumor specific antigens, differentiation antigens, embryonic antigens, cancer-testis antigens, antigens of oncogenes or mutated tumor-suppressor genes, unique tumor antigens resulting from chromosomal translocations, viral antigens, and active or immunogenic fragments thereof. In some embodiments, at least one of the one or more active agents is a tumor antigen selected from the group consisting of NY-ESO-1, SSX-2, Melan-A, tyrosinase, PRAME, and PSMA, or an immunogenic fragment thereof. In some embodiments, at least one of the one or more active agents is a hydrophobic agent. In some exemplary embodiments, the hydrophobic agent is a peptide. In some exemplary embodiments, the hydrophobic agent is an NY-ESO-1 immunogenic peptide. In some exemplary embodiments, the hydrophobic agent is NY-ESO-1₁₅₇₋₁₆₅, or an analogue thereof. In some exemplary embodiments, the NY-ESO-1₁₅₇₋₁₆₅ peptide analogue is SNvaLMWITQV (SEQ ID NO:3).

In some embodiments, a water-soluble bulking or caking agent and a surfactant is used in the formulations and methods disclosed herein. For example, in some embodiments, a sterile solution including a water-soluble bulking agent further including a surfactant is used. The water-soluble bulking agent can be mannitol, glucose, trehalose, or any such bulking agent, in some exemplary embodiments, the water-soluble bulking agent is mannitol. The surfactant can be polysorbate 80 or Triton X-100, or any such surfactant.

In some embodiments, the methods described herein further comprise a step for improving long-term stability of a hydrophobic agent or an agent having low, minimal or poor solubility in aqueous media against oxidative degradation comprising storing the lyophilized product or cake under an inert gas. In some embodiments the inert gas is nitrogen.

Embodiments of the disclosure relate to methods for preparing an injectable formulation wherein the methods include providing a sterile lyophilized cake comprising one or more active agents and a water-soluble bulking agent, wherein at least one of the one or more active agents is water-insoluble; and disintegrating the lyophilized cake in a parenterally acceptable liquid to or a liquid suspension of fine particles for administration to a patient. In some embodiments, the liquid suspension of fine particles formed is syringeable. In some embodiments, at least one of the one or more active agents is a hydrophobic agent or an agent having low, minimal or poor solubility in water. In some embodiments, at least one of the one or more active agents is both water-insoluble and lipid-insoluble.

Some embodiments of the disclosure relate to methods for delivering an injectable formulation prepared as described herein. The methods include obtaining a lyophilized cake comprising one or more active agents, wherein at least one of the one or more active agents is water-insoluble; disintegrating the lyophilized cake in a parenterally acceptable liquid/solvent to form an injectable/syringeable liquid suspension of fine particles, wherein the suspension is prepared within minutes such as 5 minutes, or 10 minutes, or 20 minutes, or 30 minutes, or 60 minutes, or 90 minutes, or 120 minutes, or 180 minutes, or 240 minutes, or 300 minutes, or 360 minutes, or 480 minutes, or at least 720 minutes prior to administration to a patient. In some embodiments, the liquid suspension of fine particles is stable, that is, the particles do not agglomerate to the point that the suspension is no longer syringeable. In some embodiments, the liquid suspension of fine particles is stable for 0.5 hours, or 1 hour, or 4 hours, or 6 hours, or 8 hours, or 12 hours, or 18 hours, or 24 hours at room temperature. In some embodiments, the suspension is stable for 6 hours at room temperature. In some embodiments, the liquid suspension of fine particles includes particles in size of less than 5 microns, or less than 10 microns, or less than 20 microns, or less than 25 microns, or less than 30 microns, or less than 60 microns, or less than 90 microns, or less than 100 microns. In some further embodiments, the fine particles include particles that are also at least 1 microns, or at least 5 microns, or at least 10 microns, or at least 20 microns, or at least 25 microns, or at least 30 microns, or at least 50 microns. In some embodiments, the liquid suspension of fine particles includes particles less than 30 microns. In some exemplary embodiments, the liquid suspension of fine particles of an NY-ESO-1 peptide (or peptide analogue) formulation includes particles 20 to 30 microns in size. The injectable formulations of fine particle suspensions disclosed can be adapted for administration or delivery to a subject intradermally, intraperitoneally intramuscularly, subcutaneously, or intranodally to the lymphoid organs (e.g., lymph nodes), but is not necessarily limited to such, and excludes administration or delivery intravenously. In some embodiments, the formulations disclosed herein are formulated or prepared for administration directly to the lymphatic system. In some embodiments, administration directly to the lymphatic system is intranodal administration.

Some embodiments of the disclosure relate to injectable formulations prepared according; to the methods and processes described herein. In some embodiments, a lyophilized cake comprising one or more active agents and a water-soluble bulking agent comprising a surfactant, wherein at least one of the one or more active agents is water-insoluble, is provided. In some embodiments, at least one of the one or more active agents is hydrophobic or an agent having low, minimal or poor solubility in water. In some embodiments, at least one of the one or more active agents is both water-insoluble and lipid-insoluble. In some embodiments, the lyophilized cake is capable of being disintegrated in a parenterally acceptable liquid/solvent to form an injectable/syringeable liquid suspension of fine particles for administration to a subject.

Some embodiments disclosed relate to a process is for preparing an injectable formulation comprising: the steps of: a) providing one or more active agents solubilized with a parenterally unacceptable volatile lyophilizable solvent, wherein at least one of the one or more active agents is water-insoluble; b) forming a solution of a water-soluble bulking agent and a surfactant; c) separately sterile filtering each of the resultant solutions of step a) and b); d) mixing the sterile solutions slowly, such as by gently or slowly stirring or gently shaking, and the like, or by any other means that prevents agglomeration and/or aggregation and/or precipitation of the water-insoluble agent; e) lyophilizing the mixed solution to produce a lyophilized cake; and f) disintegrating the lyophilized cake with a parenterally acceptable liquid. In some embodiments, at least one of the one or more active agents is a hydrophobic agent. In some embodiments, at least one of the one or more active agents is a tumor antigen selected from the group consisting of NY-ESO-1, SSX-2, Melan-A, tyrosinase, PRAME, and PSMA, or an immunogenic fragment thereof. In some exemplary embodiments, the at least one hydrophobic agent is an NY-ESO-1 immunogenic peptide. In some exemplary embodiments, the at least one hydrophobic agent is NY-ESO-1₁₅₇₋₁₆₅ peptide, or an analogue thereof. In some exemplary embodiments, the NY-ESO-1₁₅₇₋₁₆₅ analogue is SNvaLMWITQV (SEQ ID NO:3). In some embodiments, the parenterally unacceptable volatile lyophilizable solvent is acetic acid, hydrochloric acid, or ammonium hydroxide. In some exemplary embodiments, the water-soluble bulking agent includes mannitol, but is not necessarily limited to such. In some embodiments, the bulking agent further includes a surfactant. In some embodiments, the lyophilized cake is disintegrated in a parenterally acceptable solvent for injection. Exemplary parenterally acceptable solvents include water, a sodium chloride solution, or a phosphate buffer, or the like. In some embodiments, the disintegrated lyophilized cake forms a stable suspension of fine particles for administration to a patient or subject in need thereof. In some embodiments, the suspension is stable to 0.5 hours, or 1 hour, or 4 hours, or 6 hours, or 8 hours, or 12 hours, or 18 hours, or 24 hours at room temperature. In some embodiments, the suspension is stable for 6 hours at room temperature. In some embodiments, that suspension is prepared in a pharmacy, for example, a hospital or clinic pharmacy. In other embodiments, the suspension is prepared “at bedside,” that is in the presence of the patient or subject. In some embodiments, the formulations disclosed herein are formulated or prepared for administration directly to the lymphatic system of the patient or subject. In some embodiments, administration directly to the lymphatic system is intranodal administration.

In some embodiments, the disclosure relates to a kit comprising an effective amount of a lyophilized cake, prepared according to the methods and processes described above; a parenterally acceptable solvent/liquid for preparing a suspension of fine particles of the active agent from lyophilized cake prior to administration; and instructions for administering the suspension to a subject/patient in need thereof. In some embodiments, the kit includes water for injection, a sodium chloride solution, or a phosphate buffer as the parenterally acceptable solvent. In some embodiments, the lyophilized cake includes one or more active agents and a water-soluble bulking agent and a surfactant, wherein at least one of the one or more active agents is water-insoluble. In some embodiments, the water-insoluble agent is hydrophobic or an agent having low, minimal or poor solubility in water. In some embodiments at least one of the one or more active agents is both water-insoluble and lipid-insoluble. In some exemplary embodiments, at least one of the one or more active agents is a tumor antigen selected from the group consisting of NY-ESO-1, SSX-2, Melan-A, tyrosinase, FRAME, and PSMA, or an immunogenic fragment thereof. In some exemplary embodiments, the at least one hydrophobic active agent is an NY-ESO-1 immunogenic peptide, such as, for example, An NY-ESO-1₁₅₇₋₁₆₅ peptide, or an analogue thereof, such as, for example, SNvaLMWITQV (SEQ ID NO:3). In some embodiments, each component of the kit is packaged in separate containers. In some embodiments, the lyophilized cake is provided along with reagents for disintegrating the cake to form a syringeable fine particle suspension. The container can be a syringe, an ampule, a vial, or the like. In some embodiments, the kit includes a syringe. In some embodiments, the kit includes an ultrasonically opaque needle.

The instant disclosure represents a significant advancement over the recurring challenges in the development of pharmaceuticals, particularly injectable formulations comprising an active agent that is water-insoluble. The methods and processes described herein for preparing a syringeable/injectable formulation comprising at least one water-insoluble active agent as a suspension of fine particles suitable for administration to a subject, inter alia, (a) allow for aqueous liquids to be used in administration of the water-insoluble active agent, (b) circumvent the need for creating a suspension prior to filling the product; (c) do not require generating a suspendable powder by means of spray drying, crystallization, precipitation or milling, all which require complex aseptic processing equipment; (d) do not require keeping a liquid suspension, once formed stable for long periods (for example, days, months, years); and (e) result in final products that allow for administration of reliable, and reproducible amounts of the water-insoluble active agent. The suspension of fine particles, suitable for administration to a subject, prepared as by the methods and processes of the disclosure can improve the overall bioavailability of a water-insoluble active agent.

The instant disclosure provides a method of formulation specifically to formulate one or more water-insoluble active agents by dissolution of the water-insoluble agent(s) utilizing a parenterally unacceptable volatile lyophilizable solvent (such as, for example, acetic acid; capturing the dissolved state of the water-insoluble agent, in the presence of a bulking agent, and optionally, a surfactant, by freezing; and subsequently removing the parenterally unacceptable volatile solvent by lyophilization, thereby leaving the water-insoluble molecules dispersed in a lyophilized cake (i.e., finely dispersed). The lyophilized cake can be readily disintegrated with a parenterally acceptable solvent to obtain a suspension of fine particles suitable for administration to a subject, as needed. Because the suspension of fine particles is only obtained upon suspension with a parenterally acceptable solvent, the requirement for stability of the suspension is therefore reduced to only what is necessary to keep the active agent finely dispersed long enough to deliver or administer to a subject, in contrast to the situation where the suspension itself is the form in which the product is stored and shipped. The instant disclosure provides a simpler methodology and process than presently employed in the art to produce most other injectable fine particle suspensions in that the particles can form spontaneously rather than, for example, relying upon a milling or other similar process. The methods and processes disclosed herein further improve the efficacy of active agents that are water-insoluble, for use as injectables in a vaccine or immunization regimen by improving solubility and stability of the water-insoluble active agent and thereby the formulation comprising such.

In an exemplary manner, the disclosure describes results based on formulations containing suspension of fine particles of a peptide derived from the sequence of NY-ESO-1, a water-insoluble and hydrophobic agent, with the suspension formulation comprising particles 20 to 30 microns in size. In some exemplary embodiments, the disclosure relates to a method for preparing an injectable formulation of the NY-ESO-1 hydrophobic peptide that is suitable for administration to a subject (e.g., a human patient) in need thereof. In some exemplary embodiments, the hydrophobic agent is the peptide NY-ESO-1₁₅₇₋₁₆₅ or an analogue thereof. In some exemplary embodiments, the NY-ESO-1₁₅₇₋₁₆₅ analogue is SNvaLMWITQV (SEQ ID NO:3), wherein Nva indicates norvaline. Employing the methods and processes of the disclosure in some examples, an NY-ESO-1 peptide was completely solubilized in acetic acid (87.5%), a parenterally unacceptable volatile lyophilizable solvent, and sterile filtered to remove particulates that could serve to seed aggregation and/or precipitation. Separately, a solution of a water-soluble bulking agent, mannitol, with 10% polysorbate 80 was prepared and sterile filtered. The mannitol/polysorbate solution was added by controlled (very slowly with constant stirring) mixing to the NY-ESO-1 peptide solution. The combined (bulk) solution containing NY-ESO-1 peptide, acetic acid, mannitol, and polysorbate 80 was lyophilized whereby the parenterally unacceptable volatile lyophilizable solvent, acetic acid, was removed to produce a lyophilized cake. The lyophilized cake was evaluated and found to be stable over at least a three-month period. A syringeable liquid fine panicle suspension of NY-ESO-1 peptide formulation was obtained by disintegrating the lyophilized cake in a suspension buffer containing 50 mM sodium phosphate (pH 8.0) in the presence or absence of 0.5% polysorbate 80. Upon disintegration of the NY-ESO-1 lyophilized cake with a parenterally acceptable suspension buffer the suspension was stable for at least six hours at room temperature. The syringeable/injectable suspension of fine particles was found to be suitable for administration, for example intranodally, to a subject (see Examples 8-9). The NY-ESO-1 peptide suspension administered intranodally as a suspension of fine particles was able to induce a CTL response, detected by ELISPOT and chromium release assays (see Examples 8-9).

Embodiments of the disclosure relate to methods and processes for preparing an injectable formulation comprising one or more active agents. In some embodiments, at least one of the one or more active agents is water-insoluble. In some embodiments, the at least one of the one or more active agents is hydrophobic. In some embodiments, at least one of the one or more active agents is an agent having low, minimal, poor or no solubility in water. In some embodiments, at least one of the one or more active agents is water-insoluble and also lipid-insoluble (i.e., having low, minimal, poor or no solubility in lipids). An active agent of the disclosure can include, in a non-limiting manner, a pharmaceutical composition, a synthetic compound, and an organic macromolecule. An active agent can be an immunogenic, a therapeutic, a prophylactic, and/or a diagnostic molecule. An active agent can be a natural compound or an isomer, analogue, or derivative thereof. In some embodiments, an active agent can be a small molecule, a protein, or a peptide.

Peptides, proteins, and polypeptides are chains of amino acids linked by peptide bonds. Peptides are generally considered to be less than 30 amino acid residues in length, but can be longer. Proteins are generally considered to contain more than 30 amino acid residues. The term “polypeptide,” as used herein, can refer to a peptide, a protein, or any other chain of amino acids of any length containing multiple peptide bonds, though generally containing at least 10 amino acids. For simplicity and convenience, the terms “amino acid residue” and “amino acid” are used interchangeably.

In some embodiments, an active agent employed in the disclosed methods, processes, and formulations, can include an antigen or an immunogenic fragment thereof. There are many antigens, epitopes of which can be recognized by T cells in an MHC-restricted manner, for which manipulation of an immune response directed against the antigen has therapeutic or prophylactic benefit. Such antigens include tumor associated antigens, which refer to antigens associated with a malignant or non-malignant cancer. These antigens can be present in a cancerous or neoplastic cell or can be associated with non-cancerous cells of the tumor, such as tumor neovasculature, or other stromal cells within the tumor microenvironment. Accordingly, in some embodiments, active agents for use in the methods and processes disclosed herein, can include, but are not necessarily limited to cancer-testis antigens such as, for example, SSX-2, NY-ESO-1 and PRAME; differentiation antigens such as, for example, MART-1/MelanA (MART-I), gp100 (Pmel 17), tyrosinase, PSMA, TRP-1, TRP-2; and tumor-specific multilineage antigens such as, for example, MAGE-1, MAGE-3, BAGE, GAGE-1, GAGE-2, p15; over expressed embryonic antigens such as, for example, CEA; over expressed oncogenes and mutated tumor-suppressor genes such as, for example, p53, Ras, HER-2/neu; unique tumor antigens resulting from chromosomal translocations such as, for example, BCR-ABL, E2A-PRL, H4-RET, IGH-IGK, MYL-RAR; and viral antigens, such as, for example, the Epstein Barr virus antigens EBVA and the human papillomavirus (HPV) antigens E6 and E7. Other active agents can include, for example: TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, β-Catenin, CDK4, Mum-1, p15, p16, 43-9F, 5T4, 791Tgp72, alpha-fetoprotein, β-HCG, BCA225, BTAA, CA 125, CA 15-3\CA 27.29\BCAA, CA 195, CA 242, CA-50, CAM43, CD68\KP1, CO-029, FGF-5, G250, Ga733\EpCAM, HTgp-175, M344, MA-50, MG7-Ag, MOV18, NB/70K, NY-CO-1, RCAS1, SDCCAG16, PLA2, TA-90\Mac-2 binding protein\cyclophilin C-associated protein, TAAL6, TA072, TLP, and TPS. Antigens or immunogenic fragments thereof useful in the disclosed methods and processes also include those found in infectious disease causing organisms, such as, for example, structural and non-structural viral proteins. Potential target microbes contemplated for use in the disclosed formulations, processes and methods, include without limitation, hepatitis viruses (e.g., B, C, and delta), herpes viruses, HIV, HTLV, HPV, EBV, and the like. A general term for these antigens, which are recognized or targeted by the immune response, is target-associated antigen (TAA). Such active agents are disclosed in the literature and/or available commercially.

In some embodiments, an active agent or agents can include a peptide antigen, such as an epitope of a larger antigen, i.e., a peptide having an amino acid sequence corresponding to the site on the larger molecule that is presented by MHC/HLA molecules and can be recognized by T cell receptor. Such peptide antigens as contemplated herein can be of 8-15 amino acids in length, or more typically 8-10 amino acids in length. Examples of such peptides are discussed, for example, in U.S. Pat. Nos. 5,747,269 and 5,698,396; and PCT Application Number PCT/EP95/02593 (published as WO 96/01429), filed Jul. 4, 1995, entitled METHOD OF IDENTIFYING AND PRODUCING ANTIGEN PEPTIDES AND USE THEREOF AS VACCINES and PCT Application No. PCT/DE96/00351 (published as WO 96/27008), filed Feb. 26, 1996, entitled AGENT FOR TREATING TUMOURS AND OTHER HYPERPLASIA, each of which is incorporated herein by reference in its entirety.

Active agents also contemplated for use in embodiments disclosed herein, include peptides identified by the method disclosed in, for example, U.S. patent application Ser. No. 09/560,465, filed Apr. 28, 2000, U.S. patent application Ser. No. 11/683,397 (U.S. Publication 2007/0269464) filed Mar. 7, 2007, U.S. patent application Ser. No. 10/026,066 (published as US 2003/0215425 A1), filed on Dec. 7, 2001, and U.S. patent application Ser. No. 10/005,905, filed on Nov. 7, 2001, each entitled “EPITOPE SYNCHRONIZATION IN ANTIGEN PRESENTING CELLS,” (incorporated herein by reference in its entirety) including those that are apparent presently or in the future. Additional peptides, and peptide analogues that can be employed in embodiments herein disclosed, include those for example, in U.S. Provisional Application No. 60/581,001, filed on Jun. 17, 2004 and U.S. patent application Ser. No. 11/156,253 (published as US 2006/0063913), filed Jun. 17, 2005, both entitled “SSX-2 PEPTIDE ANALOGS;” and U.S. Provisional Application No. 60/580,962, filed Jun. 17, 2004 and U.S. patent application Ser. No. 11/155,929 (published as US 2006/0094661), filed Jun. 17, 2005, both entitled “NY-ESO PEPTIDE ANALOGS;” U.S. patent application Ser. No. 11/455,278 (now U.S. Pat. No. 7,511,119), U.S. patent application Ser. No. 11/454,633 (now U.S. Pat. No. 7,511,118), U.S. patent application Ser. No. 11/454,300 (now U.S. Pat. No. 7,605,227) each filed on Jun. 16, 2006, and each entitled “EPITOPE ANALOGS;” each of which is incorporated herein by reference in its entirety, U.S. patent application Ser. No. 09/561,571, filed on Apr. 28, 2000 and entitled “EPITOPE CLUSTERS;” U.S. patent application Ser. No. 10/094,699 (now U.S. Pat. No. 7,252,824), filed on Mar. 7, 2002 and entitled “ANTI-NEOVASCULATURE PREPARATIONS FOR CANCER;” U.S. patent application Ser. No. 10/117,937 (published as US 2003/0220239 A1), filed on Apr. 4, 2002, U.S. patent application Ser. No. 10/657,022 (published as US 2004/0180354 A1), filed on Sep. 5, 2003, and PCT Application No. PCT/US2003/027706 (published as WO 04/022709A2) filed Sep. 5, 2003, all entitled “EPITOPE SEQUENCES;” and U.S. Pat. No. 6,861,234; each of which is incorporated herein by reference in its entirety.

In some embodiments, the one or more active agents can include specific antigenic combinations that are beneficial in directing an immune response against particular cancers as disclosed, for example, in U.S. Provisional No. 60/479,554, filed on Jun. 17, 2003, and U.S. patent application Ser. No. 10/871,708 (published as US 2005/0118186), filed on Jun. 17, 2004; U.S. patent application Ser. No. 11/155,288 (published as US 2006/0008468) filed Jun. 17, 2005; U.S. patent application Ser. No. 11/323,049 (published as US 2006/0159694 A1), filed Dec. 29, 2005; U.S. patent application Ser. No. 11/323,964 (published as US 2006/0159689) filed Dec. 29, 2005; and PCT Patent Application No. PCT/US2004/019571, filed Jun. 17, 2004, all entitled “COMBINATIONS OF TUMOR-ASSOCIATED ANTIGENS IN VACCINES FOR VARIOUS TYPES OF CANCERS,” each of which is incorporated herein by reference in its entirety.

In some embodiments, active agents include anticancer agents, such as, but not necessarily limited to, camptothecin, taxol, O(6)-benzylguanine, paclitaxel, docetaxel, amphotericin B, and the like. In some embodiments, active agents include bacterial cell membrane proteins, transmembrane protein domains and signal peptides that can be attached to other drags to target various membranes and intracellular compartments, but are not necessarily limited to such.

Accordingly, in some embodiments disclosed herein, the one or more active agent is water-insoluble. In some embodiments, at least one of the one or more active agents is hydrophobic or an agent having low, minimal, poor or no solubility in water. Such water-insoluble or hydrophobic agents can include, for example, small molecules, proteins, or peptides having low, minimal, poor or no water solubility due to their amino acid composition or sequence and are therefore insoluble or unstable (e.g., they tend to precipitate out of solution over time (and with transport) in aqueous media. In some embodiments, the water-insoluble or hydrophobic agent is a peptide. Amino acid residues that influence the solubility or dissolution of molecules such as proteins, peptides, and polypeptides include alanine, valine, norvaline, leucine, isoleucine, phenylalanine, proline, methionine, tyrosine, and tryptophan, in a non-limiting manner. In addition, other peptide sequences rich in amino acids such as glutamine or asparagine can also be very insoluble. In addition, a peptide of a given amino acid composition, when compared to a peptide of the same amino acid composition but a different sequence, can be entirely different in that one can be readily soluble and the other extremely insoluble in aqueous media. Accordingly, the water-insoluble or hydrophobic active agent can contain at least one or more hydrophobic amino acid residues which contributes to its poor solubility, in aqueous media. In addition, intrinsic water solubilities (i.e. water solubility of the un-ionized form) for hydrophobic agents are less than 1% by weight, and typically less than 0.1% or 0.01% by weight. Hydrophobic agents, as contemplated herein, can include in a non-limiting manner, therapeutic agents such as, for example, aromatic compounds, analgesics, anti-inflammatory agents, anti-bacterial agents, anti-viral agents, anti-neoplastic agents, hydrophobic immunosuppressants, and mixtures thereof. Salts, isomers and derivatives of the above-listed hydrophobic active agents can also be used, as well as combinations and mixtures thereof. The active agents can include non-hydrophobic and non-peptide agents having low or poor solubility in aqueous media.

In some exemplary embodiments, the at least one water-insoluble or hydrophobic active agent is a hydrophobic peptide, such as, for example, an NY-ESO-1 immunogenic peptide addle an analogue thereof. NY-ESO-1 is a cancer-testis antigen found in a wide variety of tumors and is also known as CTAG-1 (Cancer-Testis Antigen-1) and CAG-3 (Cancer Antigen-3). NY-ESO-1, a tumor-associated antigen (TuAA), and is disclosed in U.S. Pat. No. 5,804,381, entitled ISOLATED NUCLEIC ACID MOLECULE ENCODING AN ESOPHAGEAL CANCER ASSOCIATED ANTIGEN, THE ANTIGEN ITSELF, AND USES THEREOF, which is hereby incorporated by reference in its entirety. Examples of NY-ESO-1 peptides and peptide analogues are disclosed in U.S. Provisional Application No. 60/580,962, filed Jun. 17, 2004 and U.S. patent application Ser. No. 11/155,929 (published as US 2006/0094661), filed Jun. 17, 2005, both entitled “NY-ESO PEPTIDE ANALOGS;” U.S. Pat. No. 5,804,381; U.S. Patent Publication Nos. US 2005/0118186, 2006/0008468, 2006/0159694, and 2006/0159689; and PCT Patent Application No. PCT/US2004/019571, as disclosed supra, each of which is hereby incorporated by reference in its entirety. This peptide, being very hydrophobic or water-insoluble, is poorly soluble in aqueous media. The poor solubility of NY-ESO-1 immunogenic peptide, and its analogues, is attributed to the presence of the hydrophobic amino acid residues, such as norvaline, leucine, isoleucine, methionine and tryptophan in its structure. These attributes cause the peptide and analogues therefrom to be insoluble in most solvents containing an appreciable amount of water (for example, 1% to 25% by weight) and, once dissolved in primarily non-aqueous solvents, to come out of solution as gels or precipitates upon standing or if the water content is increased.

In addition, the presence of methionine and tryptophan amino acid residues in the NY-ESO-1 structure leads to oxidative degradation which affects the stability of this peptide. Glutamine, which is also found in the NY-ESO-1 structure, poses another potential degradation pathway due to its deamidation attribute. The methods and processes for making a formulation as described herein can prevent, decrease, reduce or minimize oxidative degradation of an active agent in a formulation thereby prolonging the shelf-life and stability of the active agent.

In some embodiments, the one or more active agents can be both water-insoluble and lipid-insoluble (i.e., having low, minimal, poor or no solubility in lipids); and can include the NY-ESO-1₁₅₅₇₋₁₆₅ peptide or analogues thereof described herein, plus one or more immunogenic fragments of other antigens such as Tyrosinase₁₋₉, but are not necessarily limited to such. In some embodiments relating to compositions comprising more than one active agent, the additional active agents contemplated herein include agents that are hydrophilic, lipophilic, amphiphilic, water-soluble, lipid-insoluble, and the like. In some embodiments relating to compositions comprising more than one active agent, the additional active agents contemplated herein include a small molecule, protein, peptide, or a nucleic acid encoding a polypeptide or peptide.

Dissolution of the water-insoluble active agent allows for lyophilization to result in a finely dispersed active within a solid cake that upon adding a buffer or diluent, leads to a suspension of small particles resulting in increased surface area and generally improved bioavailability of the active agent. As discussed herein, the methods and processes for preparing the injectable formulation comprise dissolving one or more of the active agents in a solvent. In some embodiments herein, at least one of the one or more active agents is water-insoluble. In some embodiments, at least one of the one or more active agents is hydrophobic or has low, minimal, poor or no solubility in water. Due to the insolubility of these agents in water, a parenterally unacceptable volatile lyophilizable solvent is utilized to promote dissolution of the water-insoluble or hydrophobic agent. The parenterally unacceptable volatile lyophilizable solvent need not be a pure compound, but can be a mixture of solvents, at least one of which is a parenterally acceptable volatile lyophilizable solvent. In some embodiments, such a solvent includes a weak acid or base. In some embodiments, such a solvent includes a strong acid or base, for example. Such solvents can include those having a melting point and boiling point comparable to that of acetic acid. The volatile lyophilizable solvent can be provided at various concentrations depending on the dissolution of the one or more active agent, in particular, the water-insoluble active agent. In some embodiments, for dissolution of the one or more water-insoluble agents, a high concentration (for example, 50% to 100% of a volatile lyophilizable solvent is employed; whereas, in other instances a low or moderate concentration (for example, less than 50%) is employed. In some embodiments, the parenterally unacceptable solvent is volatile, has a relatively low boiling point, (such as, but not necessarily limited to, compared to that of water), can be substantially removed (e.g., under vacuum) without leaving a residue of the solvent at a parenterally unacceptable level. One of skill in the art can rely on guidance from, e.g., the US Pharmacopeia (USP) and the International Conference on Harmonisation of Technical Requirements for Registration of Pharmaceuticals, for Human Use (ICH) regarding acceptable levels of a residual solvent in pharmaceuticals. For example, for a class III (organic volatile) solvent, such as acetic acid, levels below 5000 ppm are considered acceptable according to ICH guidance. Such volatile solvents are substantially removable from the solution comprising the components of the injectable formulation by lyophilization as discussed herein. In some instances, in selecting an appropriate volatile lyophilizable solvent, a solution phase diagram, (well know in the art), can be utilized to determine the amount of energy needed for sublimation of a solvent. Such volatile solvents can be parenterally unacceptable solvents in that they are toxic, carcinogenic, or caustic and likely to cause tissue damage, at the concentration employed to dissolve one or more water-insoluble active agents.

Examples of parenterally unacceptable solvents useful in the methods and formulations disclosed herein can include, in a non-limiting manner, hydrochloric acid, hydrobromic acid, hydroiodic acid, acetic acid, formic acid, propionic acid, acetonitrile, trifluoroacetic acid (TFA), and hydroxides such as, but not limiting to, ammonium hydroxide. Additional examples of parenterally unacceptable solvents useful in the methods and formulations disclosed herein can include, in a non-limiting manner, solutions of the above-mentioned examples (hydrochloric acid, hydrobromic acid, hydroiodic acid, acetic acid, trifluoroacetic acid (TFA), and hydroxides such as, but not limiting to, ammonium hydroxide) in a lyophilizable solvent. In some embodiments, the parenterally unacceptable solvent includes solutions of non-lyophilizable solvents or co-solvents in a lyophilizable solvent, such that the otherwise non-lyophilizable component becomes lyophilizable (for instance by forming an azeoptrope with the lyophilizable component). In some embodiments, the parenterally unacceptable solvent includes a solution of non-lyophilizable parenterally acceptable solubility enhancer(s) in a lyophilizable solvent, to enhance the active drug substance's solubility in the parenterally unacceptable lyophilizable solvent, such that the non-lyophilizable enhancer is not removed upon lyophilization.

Additional solvents or co-solvents, such as ethyl acetate, ethanol, methanol, dimethyl formamide (DMF), acetone, acetonitrile, tetrahydrofuran (THF), dimethyl sulfoxide (DMSO), and combinations thereof, can also be utilized. In some embodiments, the parenterally unacceptable volatile solvent is acetic acid, hydrochloric acid, or ammonium hydroxide. In some embodiments, the parenterally unacceptable volatile solvent is a solution of acetic acid, hydrochloric acid, or ammonium hydroxide in another lyophilizable solvent. In some exemplary embodiments, the parenterally unacceptable volatile solvent is acetic acid.

Bulking agents are commonly used in the formulation of lyophilized or freeze dried cakes/products in order to chemically and physically stabilize the active agent, to create a finely dispersed form of the active agent that is more easily solubilized, and to provide an inert, easily disintegrated cake/product containing an isotonic solution of the active agent upon disintegration. In some exemplary embodiments, the bulking agent, mannitol, is used in the formulation of a lyophilized or freeze dried cake in order to chemically and physically stabilize the active agent. NY-ESO-1 peptide, to create a finely dispersed form of the active agent and a surfactant that spontaneously forms a fine particle isotonic suspension upon disintegration. Bulking agents can be sugars or polyols, therapeutic proteins, or the active agent of the formulation itself, or any other bulking agents in the literature or commercially available. Bulking agents can include, in a non-limiting milliner, for example, xylitol, lactose, sucrose, dextrose, glucose, inositol, raffinose, maltotriose mannitol, trehalose, or sorbitol, or other disaccharides. Two of the most commonly used hulking agents in injectable formulations include mannitol and lactose. In some exemplary embodiments, the hulking agent is mannitol. In some exemplary embodiments, two or more bulking agents are used in the same formulation of a lyophilized product or an injectable/syringeable formulation. In some exemplary embodiments, the bulking agent is dissolved in water and a surfactant is added to the solution which can reduce or minimize aggregation of the active agent during formulation of the cake, or within the cake which can reduce or minimize agglomeration of the particles formed after disintegration of the injectable formulation.

Surfactants contemplated for use herein include, in some embodiments, polysorbate 80, polysorbate 20, Triton X-100, lauryl glucoside, NP-40, oleyl alcohol, sorbitans (monosterate tristearate), stearyl alcohol, nonoxynols, Cremophore (RH 60 or EL), Solutol HS 15, plutonic acid, sodium dodecyl sulfate (SDS), soy lecithins, egg yolk lecithins and any surfactant in the literature or commercially available. In some embodiments, the surfactant is a nonionic surfactant. In some exemplary embodiments, the surfactant is polysorbate 80, a surfactant commonly used in parenteral formulations of proteins to reduce or minimize denaturation at the air-water interface. This surfactant can be used to prevent massive agglomeration of the particles in the suspension upon addition of the suspension buffer to the lyophilized cake. In some exemplary embodiments, two or more surfactants are used in the same formulation of a lyophilized product or an injectable/syringeable formulation.

In some embodiments, the disclosure provides sterile tittered solubilized solutions containing the components of the desired injectable formulation. Filter devices (sterile filtration techniques e.g., 0.2-μm filter system) can be used to separately filter each of the solutions containing the solubilized active agents in a parenterally unacceptable solvent and the water-soluble bulking agent with or without a surfactant to remove particulates that could later serve to seed aggregation and/or precipitation. Thereafter, the solutions can be combined by slowly or gently mixing in any manner that prevents aggregation and/or precipitation of the active agent, such as by gently or slowly stirring or shaking. In some embodiments, gently mixing is performed in a manner that prevents aggregation and/or precipitation of the hydrophobic NY-ESO-1₁₅₇₋₁₆₅ peptide. Merely by way of example, combining the solution containing the solubilized active agent(s) in a parenterally unacceptable solvent and a solution containing the water-soluble bulking, agent (with or without a surfactant) can be achieved by adding the solution containing the water-soluble bulking agent slowly to the solution containing the solubilzed active agent(s) in the parenterally unacceptable solvent, accompanied by gently stirring and/or shaking. Optionally, the solution formed by the combining step can again be sterile filtered. The parenterally unacceptable solvent, which can be selected for its volatility, can then be removed from the resulting (sterile) solution containing all of the mixed components of the formulation using lyophilization, a drying technique.

In some embodiments, preparation of a solution of one or more active agents includes dissolving the one or more active agents, wherein at least one of the active agent(s) is water-insoluble, in a volatile lyophilizable solvent and sterile filtering the solution; dissolving in an aqueous medium or solvent the aqueous/water-soluble components such as the bulking agent, and optionally, the surfactant, to form an aqueous solution, and sterile filtering the aqueous solution; adding the sterile filtered aqueous solution into the sterile filtered solution of the active agent(s) and volatile lyophilizable solvent by slowly and gently stirring to form a mixture. Optionally, the mixture of the solutions is then filtered and ready for lyophilization. The volatile lyophilizable solvent can be parenterally unacceptable. The aqueous medium or solvent can include water, a buffer (e.g., PBS), or the like. Exemplary volatile lyophilizable solvents, active agents, aqueous media or solvents, water-soluble bulking agents, and surfactants are described throughout the specification.

In some embodiments, both the active agent(s) and the bulking agent, as well as the surfactant, if any, are directly dissolved in the same solvent. The solvent can be volatile and lyophilizable. The solvent can be parenterally unacceptable. This solution is then sterile filtered and lyophilized. Exemplary solvents, active agents, water-soluble bulking agents, and surfactants are described throughout the specification.

In some exemplary embodiments, the order of combining the separately dissolved components to form a mixture is important. Thus, in some embodiments, the solution containing the hulking agent and surfactant is added slowly, with gentle mixing (e.g., by stirring and/or shaking), to the solution containing the water-insoluble active agent and volatile solvent in order to prevent the water-insoluble agent from rapidly precipitating. In some embodiments, for a highly hydrophobic active agent, a high concentration of the volatile solvent (for example, 50% to 100% acetic acid) is used for initial dissolution to form the solution containing the highly hydrophobic active agent, and the solution is prepared and filtered separately from all other solutions of dissolved components (e.g., other active agent(s), and/or the water-soluble balking agent(s), and/or the surfactant(s)). The separate dissolution and filtration of water-insoluble agent solution allows for removal of particulates that might seed precipitation, which, in addition to the volatile solvent of a high concentration, can be problematic for lyophilization by, for example, affecting or damaging the lyophilizing equipment. If multiple solvents are used to prepare the solutions containing the one or more active agents and containing the water-soluble bulking agent (and optionally, at least one surfactant), the solvents and the resultant solutions are miscible.

For a water insoluble or hydrophobic agent not requiring high concentrations of the volatile solvent for dissolution (i.e., it more readily dissolves), a solution can be prepared by dissolving the components comprising the formulation in a single aqueous preparation. That is, a water-insoluble agent, a water-soluble bulking agent, and optionally, a surfactant can be dissolved in the same solvent together, followed by sterile filtration and lyophilization. The solvent can be volatile and lyophilizable. The solvent can be parenterally unacceptable. Exemplary solvents, active agents, water-soluble bulking agents, and surfactants are described throughout the specification.

In some embodiments described herein, a lyophilization method is used for drying and removing the solvent from the solubilized sterile solution thereby obtaining a sterile lyophilized cake/product. Lyophilization or freeze drying, in brevity, comprises a process in which a solvent is removed from a solution or other mixture after it is frozen and placed under a vacuum, allowing the frozen solvent to change directly from solid to vapor without passing through a liquid phase. The process consists of three separate, unique, and interdependent processes; freezing, primary drying (sublimation), and secondary drying (desorption). Methods lyophilization are disclosed in, for example “Remington: The Science and Practice of Pharmacy,” 20th Ed., Lippincott Williams &Wilkins, Baltimore, Md., pp. 802-803 (2000), which is hereby incorporated by reference in its entirety.

In some embodiments, a lyophilized cake comprising one or more water insoluble active agents that is capable of being disintegrated in a parenterally acceptable solvent to form an injectable formulation is prepared as follows. A pre-lyophilization solution is prepared, wherein the pre-lyophilization solution includes: (i) one or more active agents, wherein at least one of the one or more active agents is water-insoluble; (ii) a parenterally unacceptable volatile lyophilizable solvent; and (iii) a water soluble bulking agent. The pre-lyophilization solution can further include at least one surfactant.

In some embodiments, preparing the pre-lyophilization solution includes directly dissolving at least one of the one or more water-insoluble active agents and the water-soluble bulking agent, and optionally the at least one surfactant, in the parenterally unacceptable volatile lyophilizable solvent. The dissolving can be facilitated by gentle mixing, stirring, and/or shaking. Then the pre-lyophilization solution is sterile filtered to form a sterile pre-lyophilization solution. The sterile pre-lyophilization solution is lyophilized to produce the lyophilized cake. By lyophilizing, the parenterally unacceptable solvent is substantially removed such that the lyophilized cake is substantially free from residue of the solvent at a parenterally acceptable level. As described already, one of skill in the art can rely on guidance from, e.g., the US Pharmacopeia (USP) and the International Conference on Harmonisation of Technical Requirements for Registration of Pharmaceuticals, for Human Use (ICH) regarding acceptable levels of a residual solvent in pharmaceuticals. The one or more water-insoluble active agents are dispersed (e.g., finely and/or substantially uniformly dispersed) in the lyophilized cake. The lyophilized cake is capable of being disintegrated in a parenterally acceptable solvent to form the injectable formulation comprising a suspension of fine particles of the water-insoluble active agent.

In some embodiments, preparing the pre-lyophilization solution can include dissolving the one or more active agents in the parenterally unacceptable volatile lyophilizable solvent, thereby forming a first solution, and sterile filtering the first solution to form a first sterile solution; dissolving, the water-soluble bulking agent, and optionally, the at least one surfactant, in an aqueous solvent/medium thereby forming a second solution, and sterile filtering the second solution to form a second sterile solution; and adding the second sterile solution to the first sterile solution by controlled mixing to prevent aggregation or precipitation of the water-insoluble agent. The parenterally unacceptable volatile lyophilizable solvent (and the (sterile) first solution) is miscible with the aqueous solvent/medium (and the (sterile) second solution).

In some embodiments involving more than one active agents, each one of the active agents can be separately dissolved in a parenterally unacceptable volatile lyophilizable solvent and separately sterile filtered; and then the multiple sterile solutions containing the active agents and the second sterile solution containing the water-soluble bulking agent (and optionally, the at least one surfactant) are combined as described above to form a pre-lyophilization solution.

In some embodiments involving two or more active agents, at least one of the active agents is dissolved in one parenterally unacceptable solvent and sterile filtered, and one or more of the active agents are dissolved in a separate parenterally unacceptable solvent and sterile filtered, and then the multiple sterile solutions containing the active agents and the second sterile solution containing the water-soluble bulking agent (and optionally, the at least one surfactant) are combined as described above to form a pre-lyophilization solution.

In some embodiments involving more than one active agent, at least one of the active agents is dissolved in a parenterally unacceptable volatile lyophilizable solvent and sterile filtered to form one or more sterile solutions containing the one or more active agents, and at least one of the active agents is dissolved with the water-soluble bulking agent (and optionally, the at least one surfactant) in an aqueous solution thereby forming a second solution, and sterile filtered to form a sterile solution, and then sterile solution(s) containing at least one active agent in an unacceptable volatile lyophilizable solvent and the sterile solution containing at least one active agent and the water-soluble bulking agent (and optionally, the in least one surfactant) are combined as described above to form the pre-lyophilization solution.

Any one of the dissolving steps described herein can be facilitated by gentle mixing, stirring, and/or shaking. The pre-lyophilization solution can be sterile filtered to form a sterile pre-lyophilization solution. The sterile pre-lyophilization solution is lyophilized to produce the lyophilized cake. By lyophilizing, the parenterally unacceptable solvent(s) is/are substantially removed such that the lyophilized cake is substantially free from residue of the solvent(s) at a parenterally unacceptable level. The one or more water-insoluble active agents are dispersed (e.g., finely and/or substantially uniformly dispersed) in the lyophilized cake. The lyophilized cake is capable of being disintegrated in a parenterally acceptable solvent to form the injectable formulation comprising a suspension of fine particles of the water-insoluble active agent.

Disintegration of a lyophilized product and formation of the suspension can be influenced by a number of parameters that can affect the stability, effectiveness, and production of a suspension of fine particles. Such parameters include: porosity; solid-state form of the cake; degree of crystallinity; cake wettability (the ability of the cake to imbibe the solvent—suspension or disintegration buffer); formulation factors such as the moisture content of the lyophilized cake; physical and chemical degradation due to excess moisture; foaming, which can lead to protein denaturation and decrease in activity; and gel formation (gelatinous clump) upon contact between the cake and a parenterally acceptable solvent (e.g., suspension or disintegration buffer). Other parameters that can influence the formulation can include, the method of mixing during suspension (e.g., shaking, rolling etc.); treatments of the container, such as a glass vial, to prevent the formulation from sticking to it, which can impede the wetting of the lyophilized cake and hence its disintegration or suspension; headspace of the glass vial, which can influence the volume of the suspension; and the temperature of the suspension.

Accordingly, in some embodiments, the disclosure provides a lyophilized cake/product that can be readily disintegrated (i.e., for immediate use, in some instances at a patient's bedside), in a parenterally acceptable solvent to form an injectable/syringeable suspension of fine particles including one or more water-insoluble active agents for administration to the patient. The terms “parenterally acceptable” or “pharmaceutically acceptable” refer to those characteristics that make a drug formulation suitable in that it does not cause an allergic, toxic, or other undesirable reaction, and therefore practical for administration to a subject (e.g., a human, or another mammal). They are also physiologically acceptable when introduced into the body by a suitable route of administration; this implies, for example, that if the “parenterally acceptable” or “pharmaceutically acceptable” compositions cause adverse side effects (e.g., tissue damage, toxicity and/or carcinogenicity), then the problems caused by those adverse effects are outweighed by the immunogenic, therapeutic or prophylactic benefits of the compositions and/or treatment. For example, when lyophilized products are disintegrated with low ionic strength solutions, the resultant preparation can cause erythrocyte aggregation. Depending on the salt content of the lyophilized product, use of a saline solution, such as normal saline solution (0.9% NaCl), to disintegrate the lyophilized product could result in the suspension being hypertonic which can have undesirable side effects upon injection. Therefore, in some instances, sterile water for injection (sWFI) can be a more efficacious or desired disintegration liquid as its use can avoid the adverse effects of high ionic strength liquids.

Parenterally acceptable solvents used to disintegrate a lyophilized cake described herein into an injectable dosage form, include, but are not necessarily limited to, solubilizers, wetting agents, buffering agents, chelating agents, diluent, fillers, dispersion media, surfactants, antioxidants, preservatives (e.g., antibacterial agents, antifungal agents), isotonic agents, salts, drag stabilizers, binders, and such like materials and combinations thereof. Except insofar as any conventional liquid is incompatible with the active agent, its use in the parenteral/pharmaceutical formulations or compositions disclosed herein is contemplated. In some embodiments, a lyophilized cake can be disintegrated with a sterile solution such as, for example, but not necessarily limited to, 5% dextrose solution, normal saline, phosphate buffer, or sterile or bacteriostatic water for injection before administration. In some embodiments, the lyophilized cake can be disintegrated in phosphate buffer. In some embodiments, the lyophilized cake can be disintegrated at a patient's bedside for immediate use. In some embodiments, the lyophilized cake can be disintegrated, for example, up to 360 minutes before administration to a subject. For example, in some embodiments, the lyophilized cake is disintegrated 5 minutes, or 10 minutes, or 20 minutes, or 30 minutes, or 40 minutes, or 60 minutes, or 120 minutes, or 180 minutes, or 240 minutes, or 300 minutes, or longer than 300 minutes, prior to administration to a subject.

In some embodiments, wherein the formulation/composition is in a liquid form, the liquid can be a solvent or dispersion medium comprising, but not necessarily limited to, water-soluble solvents such as, but not necessarily limited to: water, ethanol, polyol (e.g., glycerol, propylene glycol, liquid polyethylene glycol (e.g., PEG 300 or PEG 400), etc.), glycerin, N-methyl-2-pyrrolidone (NMP), dimethylacetamide (DMA); or lipids (e.g., triglycerides, vegetable oils, liposomes), phospholipids, cyclodextrins and combinations thereof. Fluidity can be maintained, for example, by the use of a coating, such as lecithin; by the maintenance of the required particle size by dispersion in carriers such as, for example, liquid polyol or lipids; by the use of surfactants such as, for example, hydroxypropylcellulose in addition to those described elsewhere herein; or combinations thereof. In some embodiments, one or more isotonic agents, such as, for example, sugars, sodium chloride, or combinations thereof, are included. In addition to those described above, water miscible organic injectable solvents and surfactants as described above can be used. Bulking agents can be, for example, mostly non-ionizing and include those described above. Polymers can include, for example, dextral, polyvinyl alcohol (PVA), hydroxypropyl methylcellulose (HPMC), gelatin, polyethylene glycol (PEG), polyvinylpyrrolidone (PVP), and the like. Parenteral formulations can also contain buffering agents. Buffering agents include agents that maintain a solution pH in an acceptable range. Exemplary buffering agents include, for example, acetate, citrate, glycine, histidine, phosphate (sodium or potassium), and diethanolamine.

Embodiments of the disclosure relate to providing a lyophilized cake prepared by the methods and processes described herein, comprising one or more active agents, wherein at least one of the one or more active agents is a hydrophobic molecule and wherein the lyophilized cake is capable of being disintegrated in a parenterally acceptable solvent to form a syringeable liquid suspension of fine particles for administration to a subject. In some embodiments, the administered formulation is capable of inducing, enhancing, priming, initiating, prolonging, maintaining, amplifying, augmenting, or boosting a T cell response.

According to the methods and processes disclosed herein, the syringeable formulation can be for delivery to a subject by any method for administering an injectable formulation to a subject excluding intravenous administration. Hence, an injectable formulation as described herein can be adapted for administration to a subject intradermally, intraperitoneally, intramuscularly, mucosally, subcutaneously, and intranodally (i.e., to lymph nodes), but is not necessarily limited to such. In some embodiments, the injectable formulation is suitable for administration by direct delivery to the lymphatic system, typically to secondary lymphatic organs such as lymph nodes or their associated vessels. In some embodiments, the injectable formulation is administered to a lymphatic vessel, organ, or node. In some embodiments, the injectable formulation is adapted for delivery into the lymphatic system of the subject, wherein the formulation is for delivery to a lymph vessel, lymph node, the spleen, tonsils, or other appropriate portion of the lymphatic system. In some embodiments, the injectable formulation is administered by direct delivery to a lymph node, such as an inguinal or axillary node, by way of a catheter or needle to the node and maintaining the catheter or needle in place throughout the delivery. The syringeable formulation of a suspension of fine particles disclosed is not recommended for intravenous administration as it may cause damage to blood vessels at or near the injection site, and/or precipitation of insoluble components that can lead to occlusion (blockage) of the blood vessels, which can result in damage to the heart, brain, or other organs.

The injectable formulation(s) of a suspension of fine particles disclosed herein can be delivered by bolus injection with a hypodermic syringe, as in the examples below, or by other similarly functional devices for administration. This syringeable suspension of fine particles can include particles of 20 to 30 microns in size which can dictate the gauge of the needle used for delivery of an injectable formulation of the disclosure. Other methods of delivery/administration can include infusion, for example subcutaneously or directly into the lymphatic system by a delivery vehicle, such as, for example, a pump. In some embodiments, the delivery vehicle is external to the subject but contains a means (e.g., a needle or catheter) to deliver the injectable formulation into the body. In some embodiments, delivery is to a lymphatic organ or area of high lymphatic flow or drainage. An advantage of a delivery vehicle is that it obviates multiple ongoing injections.

Suitable needles or catheters can be made of metal or plastic (e.g., polyurethane, polyvinyl chloride (PVC), TEFLON, polyethylene, and the like). In inserting the catheter or needle into the inguinal node for example, the inguinal node can be punctured under ultrasonographic control using, for example, a Vialon™ Insyte-W™ cannula and catheter of 24G3/4 (Becton Dickinson, USA) which is fixed using Tegaderm™ transparent dressing (Tegaderm™ 1624, 3M, St. Paul, Minn. 55144, USA). This procedure is generally done by an experienced radiologist. The location of the catheter tip inside the inguinal lymph node can be confirmed by injection of a minimal volume of saline, which immediately and visibly increases the size of the lymph node. The latter procedure allows confirmation that the tip is inside the node. This procedure can be performed to ensure that the tip has not slipped out of the lymph node. In the event that the tip does slip out of location inside the lymph node, a new catheter can be implanted.

In some embodiments, it is desirable that an effective amount of the injectable formulation as described herein be administered or delivered intranodally to a subject thereby eliciting a T cell response. Intranodal administration is disclosed, for example, in U.S. Pat. Nos. 6,994,851 and 6,977,074; PCT Patent Publication No. WO/9902183A2, each entitled “METHOD OF INDUCING A CTL RESPONSE”; U.S. patent application Ser. No. 10/871,707, (Publication No. 2005/0079152), filed Jun. 17, 2004, entitled “METHODS TO CONTROL MHC CLASS I-RESTRICTED IMMUNE RESPONSE;” and U.S. patent application Ser. No. 11/323,572, (Publication No. 2006/0165711), filed Dec. 29, 2005, entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE,” each of which is hereby incorporated by reference in its entirety. Diagnostic techniques to assess and monitor immune responsiveness with methods of immunization are discussed more fully, for example, in U.S. patent application Ser. No. 11/155,928 (Publication No. 2005/0287068), filed Jun. 17, 2005 and entitled “EFFICACY OF ACTIVE IMMUNOTHERAPY BY INTEGRATING DIAGNOSTIC WITH THERAPEUTIC METHODS,” which is incorporated herein by reference in its entirety.

In some embodiments, administration of a suspension of fine particles as an injectable formulation as described herein, is adapted in any manner compatible with the dosage of a parenteral composition, and in such amount as will be immunogenically, therapeutically or prophylactically effective. An effective amount or dose of an injectable formulation as described herein is the amount required to provide a desired response in a subject to be treated including, but not necessarily limited to: prevention, diminution, reversal, stabilization, or other amelioration of a disease or condition, its progression, or the symptoms thereof. The concentration of the suspension described herein can be easily and readily controlled by adding more or less of an appropriate and/or acceptable disintegration buffer or liquid as described elsewhere herein. The dosage of an effective amount of formulation and the dosage schedule can vary on a subject by subject basis, taking into account, for example, factors such as the weight and age of the subject, the type of disease and/or condition being treated, the severity of the disease or condition, previous or concurrent therapeutic interventions, the capacity of the immune system to respond, the degree of protection desired, the manner of administration and the like, all of which can be readily determined by the practitioner. In some embodiments the suspension of fine particles is suspended to a desired or target concentration based on a specific/intended application or need thereof. In some embodiments a desired or target concentration of the active agent can be 0.1 mg/ml, or 0.5 mg/ml, or 1 mg/ml, or 2 mg/ml or 5 mg/ml, or 10 mg/ml, or 20 mg/ml, or 30 mg/ml, but is not necessarily limited to such. In some exemplary embodiments, the desired or target concentration is 1 mg/ml.

The injectable formulations described herein can include various “unit doses.” Unit dose is defined as the dose containing a predetermined-quantity of the active agent calculated to produce a desired response in association with its administration, i.e., the appropriate route and treatment regimen. The quantity of the formulation to be administered and the particular route of administration are within the skill of those in the clinical arts. Also of importance is the subject to be treated, in particular, the state of the subject and the protection desired. A unit dose need not be administered as a bolus injection but can comprise continuous infusion over a set period of time. In some embodiments, a unit dose can comprise from 0.5 micrograms to 100 micrograms of active agent. In some embodiments, a unit dose can be from 1 microgram to 50 micrograms. In some embodiments, the unit dose can be 10 micrograms, or 15 micrograms, or 25 micrograms, or 40 micrograms, or 50 micrograms.

In some embodiments of the disclosure, the injectable formulation is administered within 0.5 hours, or 1 hour, or 2 hours, or 3 hours, or 4 hours, or 5 hours, or 6 hours, or 7 hours, or 8 hours, or 9 hours, or 10 hours or more after disintegration into a fine particle syringeable suspension. In some embodiments, the injectable formulation is administered within at least 6 hours after disintegration. In some embodiments, the injectable formulation is administered immediately after disintegration.

Any of the components, formulations and/or compositions described herein can be assembled together in a kit. The kit can be an assemblage of materials or components, including at least one of the formulations described herein. In a non-limiting example, one or more active agents or reagents for preparing an injectable formulation are provided in a kit alone, or in combination with additional agent(s). A sterile lyophilized cake or product in addition to other agents or reagents such as a parenterally acceptable solvent or disintegration buffer for preparing a syringeable formulation of the lyophilized cake or product for administration to a subject are provided in a kit. Such kits comprise one or more suitable containers for storing and dispensing the active agents or lyophilized cake or product, or reagents. In some embodiments, the kit includes, in separate suitable containers, additional agents such as, but not necessarily limited to, buffers, surfactant and the like, in some embodiments, the kit contains two or more doses of a formulation, or others component(s) described herein, with each dose provided in separate suitable containers. For example, the kit can contain 2 doses, or 3 doses, or 4 doses, or 5 doses, or 6 doses, or 7 doses, or more than 7 doses of a formulation, wherein each dose can be in a separate container. The exact nature of the components configured in the kit depends on is intended purpose.

The materials or components assembled in the kit can be provided to the practitioner stored in any convenient and suitable ways that preserve their operability and utility and can be provided at room temperature, on ice or frozen. In some embodiments of the disclosure, the formulations prepared as described herein are provided in a lyophilized cake form that is suitable and readily disintegrated in aqueous media for injection into a subject. The additional components of the kit can be provided in one or more liquid solutions, the liquid solution is an aqueous solution, with a sterile aqueous solution being an exemplary embodiment. The liquid solutions can include the parenterally acceptable solvent or disintegration buffer, provided in a syringe and/or other such like apparatus. The syringe containing the liquid or buffer can be used to disintegrate the lyophilized cake or product and for delivering or injecting the disintegrated formulation into a subject. In some embodiments, the additional components of the kit are provided as a lyophilized product. When components (e.g., reagents) are provided as a lyophilized product, the product can be disintegrated by the addition of a suitable parenterally acceptable solvent or disintegration buffer. It is envisioned that the liquid or buffer can also be provided in a separate containers.

Thus, the container can include at least one vial, ampule, syringe, test tube, flask, bottle and/or other containers, containing the formulation and/or additional components in quantities suitable for administration to the patient. The kit can also comprise a second container for containing a sterile, parenterally acceptable buffer and/or other liquid/solvent. The kit of the disclosure can typically include the materials for practicing the methods and processes of the disclosure, and any other reagent containers in close confinement for commercial sale. Irrespective of the number or type of containers, the kit(s) described herein can also comprise, or be packaged with, an instrument for assisting with the injection/administration of a formulation as described heroin, within the body of a subject. Such an instrument can be a syringe, pump and/or any such delivery vehicle. In some embodiments, the container is a syringe and the syringe comprises an ultrasonically opaque needle. Optionally, the kit can also contain other useful components, such as, buffers, pharmaceutically acceptable liquids, syringes, catheters, applicators, pipetting or measuring tools, bandaging materials or other useful paraphernalia as will be readily recognized by those of skill in the art. In some embodiments, the kit includes instructions for preparing and administering the formulation. Instructions for use typically include a tangible expression describing the technique to be employed in using the components of the kit to affect a desired outcome.

The kit and the contents therein are typically contained in suitable packaging material(s) for sale and/or shipping. Such packaging material can include, but is not necessarily limited to, material such as plastic, paper, foil, and the like, capable of holding the individual kit. Such packaging materials can include injection or blow-molded plastic containers into which the desired vials or ampules are retained. As used herein, the phrase “Packaging material” refers to one or more physical structures used to house the kit and its contents, (i.e., such as the compositions and formulations) disclosed herein and the like. The packaging material is constructed by well known methods, preferably to provide a sterile, contaminant-free environment. The packaging material generally has an external label that indicates the contents and/or purpose of the kit and/or its components.

The following embodiments and aspects thereof are described and illustrated in conjunction with compositions and methods which are meant to be exemplary and illustrative, not limiting in scope.

### EXAMPLES

The following examples are included to demonstrate embodiments disclosed herein. It is appreciated by those of skill in the art that the methodology and compositions disclosed in the examples which follow represent methodology discovered by the inventors to function well in the practice of the disclosure, and thus can be considered to constitute particular modes for its practice. However, those of skill in the art can, in light of the present disclosure, appreciate that many changes can be made in the specific embodiments which are disclosed and still obtain a like or similar result without departing from the spirit and scope of the disclosure.

### Example 1

### Materials and Methods

Studies were conducted focused on developing an injectable formulation for a hydrophobic NY-ESO-1 peptide, NY-ESO-1₁₅₇₋₁₆₅. (SEQ ID NO:1) and analogues thereof. Table 1 provides a list of sequence analogues of the peptide useful in alternative embodiments. However, it is noted that the examples are not necessarily limited to the use of such molecules. The protein and cDNA sequences of NY-ESO-1 are identified by accession number and provided in the sequence listing filed herewith.

TABLE 1
NY-ESO-1 Analogues

| SEQ | | | % Peptide |
|---|---|---|---|
| ID NO | IDENTITY | SEQUENCE** | Binding |
| 1 | NY-ESO-1 157-165 | SLLMWITQC | 59 |
| 2 | NY-ESO-1 157-165 (C165V) | SLLMWITQV | 96 |
| 3 | NY-ESO-1 157-165 (L158Nva, C165V) | S(Nva)LMWITQV | 83 |
| 4 | NY-ESO-1 157-165 (L158I, C165V) | SILMWITQV | 72 |
| 5 | NY-ESO-1 157-165 (L158V) | SVLMWITQC | 48 |
| 6 | NY-ESO-1 157-165 (S157F, C165V) | FLLMWITQV | 63 |
| 7 | NY-ESO-1 157-165 (S157F, L158I, C165V) | FILMWITQV | 67 |
| 8 | NY-ESO-1 157-165 (S157F L158V, C165V) | FVLMWITQV | 72 |
| 9 | NY-ESO-1 157-165 (S157K, C165V) | KLLMWITQV | 81 |
| 10 | NY-ESO-1 157-165 (S157K, L158V, C165V) | KVLMWITQV | 76 |
| 11 | NY-ESO-1 157-165 (S157K, L158(Nva), C165V) | K(Nva)LMWITQV | 87 |
| 12 | NY-ESO-1 157-165 (S157T, L158V, C165V) | TVLMWITQV | 65 |
| 13 | NY-ESO-1 157-165 (S157Y, C165V) | YLLMWITQV | 80 |
| 14 | NY-ESO-1 157-165 (S157Y, C165V)-NH2 | YLLMWITQV-NH2 | 37 |
| 15 | NY-ESO-1 157-165 (S157Y, I162A, C165V) | YLLMWATQV | 104 |
| 16 | NY-ESO-1 157-165 (S157Y, I162L C165V) | YLLMWLTQV | 89 |
| 17 | NY-ESO-1 157-165 (S157Y, I162N, C165V) | YLLMWNTQV | 102 |
| 18 | NY-ESO-1 157-165 (S157Y, I162T, C165V) | YLLMWTTQV | 93 |
| 19 | NY-ESO-1 157-165 (S157Y, L158(Nva), C165V) | Y(Nva)LMWITQV | 80 |
| 20 | NY-ESO-1 157-165 (S157Y, L158I, C165V) | YILMWITQV | 72 |
| 21 | NY-ESO-1 157-165 (S157Y, L158V, C165V) | YVLMWITQV | 72 |
| 22 | NY-ESO-1 157-165 (S157Y, M160A, C165V) | YLLAWITQV | 81 |
| 23 | NY-ESO-1 157-165 (S157Y, M160I, C165V) | YLLIWITQV | 69 |
| 24 | NY-ESO-1 157-165 (S157Y, M160L, C165V | YLLLWITQV | 74 |
| 25 | NY-ESO-1 157-165 (S157Y, M160N, C165V) | YLLNWITQV | 106 |
| 26 | NY-ESO-1 157-165 (S157Y, M160V, C165V) | YLLVWITQV | 72 |
| 27 | NY-ESO-1 157-165 (S157Y, Q164A, C165V) | YLLMWITAV | 85 |
| 28 | NY-ESO-1 157-165 (S157Y, Q165E, C165V) | YLLMWITEV | 78 |
| 29 | NY-ESO-1 157-165 (S157Y, Q164N, C165V) | YLLMVITNV | 81 |

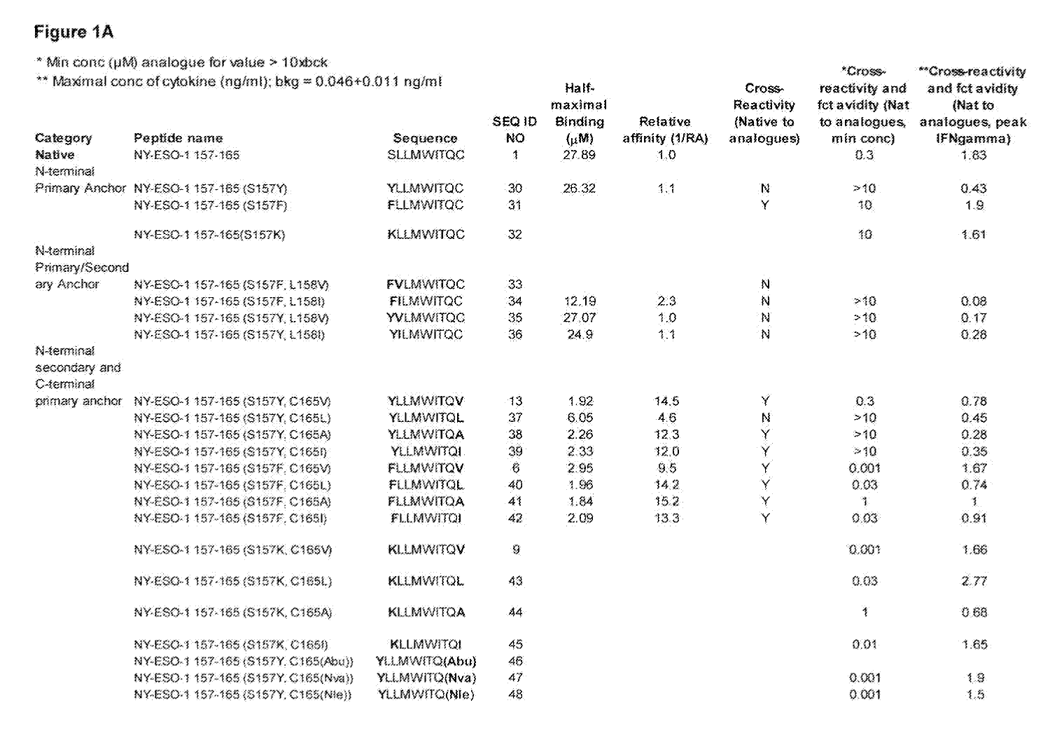

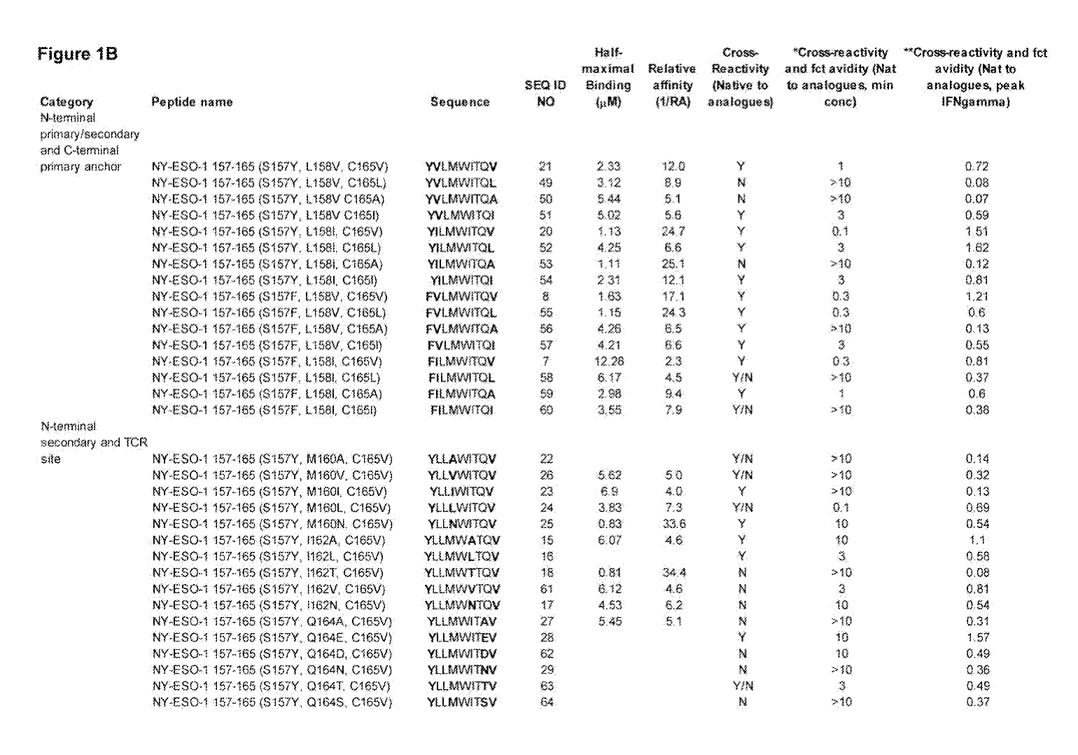

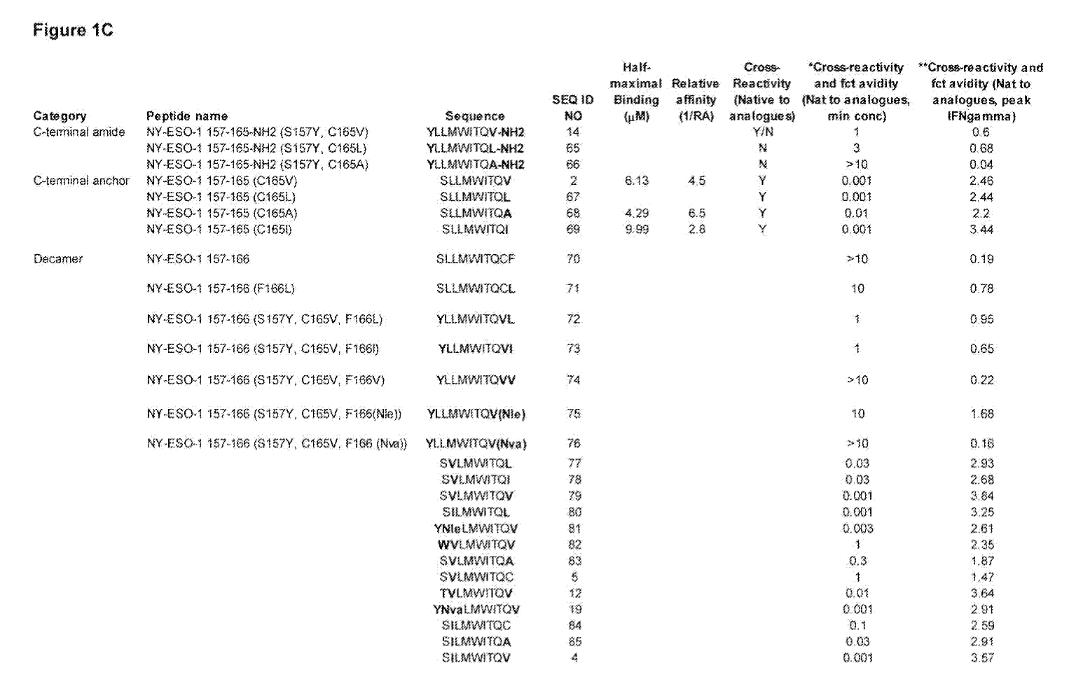

FIGS. 1A-1C provide a more detailed list of additional NY-ESO-1₁₅₇₋₁₆₅ analogues.

Materials & Reagents:—The disintegration buffer used contained phosphate buffer with or without polysorbate 80. A low peroxide USP grade polysorbate 80 was used in order to minimize oxidation of susceptible amino acids such as tryptophan, methionine and cysteine by residual peroxides, (including hydrogen peroxide). Acetic acid (volatile lyophilizable solvent), polysorbate 80 (wetting/disintegrating agent), mannitol (caking/bulking agent) and sodium phosphate (mono and dibasic; buffering agent) were purchased from Fisher Scientific, (Pittsburgh, Pa., U.S.A.). NY-ESO-1 peptide (SEQ ID NO:3) was obtained from American Peptide Company, (Sunnyvale, Calif., U.S.A.).

Buffers—Disintegration Buffer A, used in Formulation 1 (see below), consisted of 50 mM sodium phosphate buffer pH 8.0 with 0.5% polysorbate 80. Disintegration Buffer B, used in Formulation 2 (see below), consisted of 50 mM sodium phosphate buffer pH 8.0.

### Example 2

### Preparation of Pre-Lyophilization Bulk NY-ESO-1 Peptide Solution

Solutions of NY-ESO-1 peptide analogue (SEQ ID NO:3) were prepared as described below to obtain sterile pre-lyophilization bulk solutions with different polysorbate 80 concentrations.

In order to remove any possible nuclei for peptide aggregation or precipitation, all glassware was carefully washed by first soaking in chromic/sulfuric acid wash solution for an hour, followed by multiple rinses with water, and dried. The final rinse was with distilled deionized water. Following this, an appropriate amount of peptide, corrected for peptide purity and peptide content, was weighed out. To dissolve the peptide, a volume of 87.5% acetic acid was added to obtain a peptide concentration of 1.25 mg/mL and the mixture stirred until the dissolution was complete. The peptide solution was then filtered through a 0.2-micron PES (polyethersulfone) filter membrane to remove particulates that could later serve to seed aggregation/precipitation.

To prepare a pre-lyophilization bulk solution containing NY-ESO-1 peptide (0.5 mg/ml), acetic acid (35%), mannitol (4%) and polysorbate 80 (0.5%), in addition to the peptide solution described above, a solution of mannitol and polysorbate 80 was prepared separately. A specific amount of mannitol (MW=182.7 g/mol) was dissolved in water by stirring and then polysorbate 80 (10%) was added to the solution. The amounts of the components were weighed such that the final concentrations of mannitol and polysorbate 80, after addition to the peptide solution, were 4% and 0.5% respectively. The mannitol/polysorbate 80 solution was filtered through a 0.2-micron PES membrane filtration cup and added slowly, in intervals, over a few minutes, to the peptide solution while stirring gently to minimize the potential for aggregation/precipitation. Stirring after addition to the peptide solution was limited to less than 5 minutes in order to prevent peptide aggregation/precipitation.

To determine the effect of a different concentration of polysorbate 80 in the product, the above process was repeated using a second solution of mannitol and polysorbate 80 prepared such that the final concentrations of polysorbate 80 in the pre-lyophilization bulk solution was 1.0%.

### Example 3

### Determining Storage Conditions of Pre-Lyophilization Bulk Peptide Solutions

To determine the appropriate storage conditions for the pre-lyophilization bulk solutions, the solutions were stored at 5° C., 25° C., and 40° C. Storage at 5° C. resulted in peptide precipitation within minutes. In addition, the solutions were found to be more stable when stored in silanized glass than in regular type A glass. At 25° C. it was observed that the solutions could be stored for up to 2 hours in silanized glass, in the absence of shearing or shaking. Based on the observations at 5° C. and 25° C., the pre-lyophilization bulk solutions were filled (0.5 ml) into silanized vials and flash frozen in liquid nitrogen immediately after preparation, and lyophilized to a cake thereby removing the volatile acetic acid solvent. The vials were sealed wider vacuum to minimize oxidative degradation.

The lyophilized formulations were then evaluated based on several parameters which included physico-chemical stability, monitoring of moisture content of the cake (pre-suspension), and post-suspension peptide concentration, particle size, and potency. These parameters are discussed elsewhere herein and in Examples 4-6, below.

### Example 4

### Stability of the Lyophilized Product

As discussed elsewhere herein, one key aspect of a drug formulation is its short or long term physico-chemical stability, over a period of minutes, hours, weeks, or months. Therefore, based on the studies conducted with the pre-lyophilization bulk solution, Formulation 1, containing 0.5 mg/mL of peptide, 4% mannitol and 0.5% polysorbate 80; and Formulation 2, containing 0.5 mg/mL of peptide, 4% mannitol and 1% polysorbate 80, were selected for further analyses. The stability of these lyophilized formulations was evaluated according to the stability protocol presented in Table 2. The formulations were evaluated for appearance of the cake and the suspension as measured by visualization; % label claim/purity, in which the peptide concentration and presence of impurities in the composition was measured by HPLC; moisture, as measured by the Karl Fischer method which was used to determine the water content of the lyophilized cake; particle size, which was measured by light scattering; and potency by ELISPOT and Chromium release assays following immunization.

TABLE 2
Parameters of the Stability Protocol

| | |
|---|---|
| Appearance | The appearance of the cake (firm, broken, collapsed, colored) was recorded. |
| | After diluent was added, the appearance of the suspension (chunky, milky, |
| | foams, clear) was also recorded. |
| % Label | The percent label claim of the peptide was performed concomitantly with the |
| Claim/Purity | analysis of purity by high performance liquid chromatography. This assay used |
| | a reverse phase cyano column eluted with a gradient water/acetonitrile eluent |
| | with 0.1% trifluoroacetic acid and a UV detector at 225 nm to quantitate the |
| | NY-ESO-1 peptide and to detect and quantitate impurities. The peptide was |
| | quantitated in solution preparations in a single point calibration against a well |
| | characterized reference standard. The assay was capable of quantitating the |
| | peptide in the presence of impurities such as excipients or degradation products. |
| Moisture | Water content of peptides was determined through the titration of water in a |
| | buffered anhydrous solution containing sulfur dioxide and iodine, which reacts |
| | with hydrogen ions. A Karl Fischer apparatus was used for this purpose. The |
| | instrument was calibrated with a reference, such as, lactose monohydrate |
| | reference standard with known water content (e.g. 5.0%). A known amount of |
| | the peptide sample was accurately weighed and placed in the titration vessel. |
| | Replicate runs were performed for the samples and the reference. The results |
| | were reported as percent (w/w) water content. |
| Particle Size | The particle size and particle size distribution of NY-ESO-1 peptide |
| | suspensions were determined by laser light scattering. This procedure used a |
| | Sympatee HELOS laser diffraction unit with a cuvette attachment capable of |
| | measuring particles in suspension in the range of 0.5 μm-175 μm. The assay |
| | used buffers A and B described above, as appropriate, as a reference in the |
| | cuvette. The sample was then added and analyzed by the instrument once an |
| | optimal concentration of greater that 1.0% was reached. A particle distribution |
| | diagram and the X₅₀ or median (half of the particles observed are below this |
| | size) were recorded at time zero and at 3 months after storage. Particle |
| | distribution and the X₉₀ (90% of particles observed are of a size below the value |
| | reported) were recorded at 6 hours after disintegration in buffer. |
| Potency | The MHC binding kinetics of the peptide in the formulation solution was |
| | conducted by an iTopia ™ (Beckman Coulter's iTopia ™ Epitope Discovery |
| | System) assay. The principle of the method is analogous to ELISA. High |
| | throughput microtiter plates were coated with human Class I major |
| | histocompatibility complex (MHC) molecules, specifically the human leukocyte |
| | antigen HLA-A*0201. The peptide binds to the MHC molecule inducing |
| | folding of MHC. The conformational change allowed anti-HLA antibody |
| | (fluorescently labeled) to bind to the newly formed MHC-peptide complex with |
| | binding quantitated by flourometry and results reported relative to a standard. |
| | There were two parts in the binding kinetic assay. The first part was the binding |
| | assay, which characterizes instant binding to the MHC molecules by the |
| | peptide-binding measurement at time zero. It was expressed as percent |
| | binding relative to the positive control (provided by the kit manufacturer). The |
| | second part was the off-rate assay, with measured the length of time the |
| | peptide remains bound to the MHC molecule at the standard conditions (37° C.). |
| | The plate, after applying the peptide and a required overnight binding |
| | incubation at 21° C., was not immediately washed with wash buffer. The plate |
| | was removed to the 37° C. incubator and continued to be incubated. As |
| | incubation continued, peptide molecules were loosened and released from the |
| | plate (into the solution in the well). The amount of peptide remaining bound |
| | was measured at various time points. The off-rate can be calculated from the |
| | binding half-life, t_{1/2}, the length of time at which the amount of peptide bound |
| | to the MHC molecules (at 37° C.) has been reduced by 50%. |

Each of the lyophilized formulations were stored in vials and placed at 5° C., 25° C. and 40° C. for three months in order to evaluate the effect of temperature on stability. The 3-month stability vials containing lyophilized formulations of the NY-ESO-1 peptide were analyzed according to the stability protocol discussed above. Based on the peptide recovery data presented in Tables 3 and 4, both formulations appeared to be stable at 5° C. over the three-month storage period. The cake appearance was consistently firm and white for both formulations at 5° C., compared to the appearance of the cakes stored at 25° C. and 40° C. which tended to be broken. Thus, in some embodiments, a preferred storage temperature for the lyophilized formulations is 5° C. Each lyophilized cake was disintegrated in 1 mL of buffer—Buffer A (50 mM sodium phosphate buffer, pH 8.0 with 0.5% polysorbate 80) was used for Formulation 1 and Buffer B (50 mM sodium phosphate buffer, pH 8.0) was used for Formulation 2. The appearance of suspensions upon disintegration of the cake was hazy for both formulations at all storage temperatures (5° C., 25° C. and 40° C.).

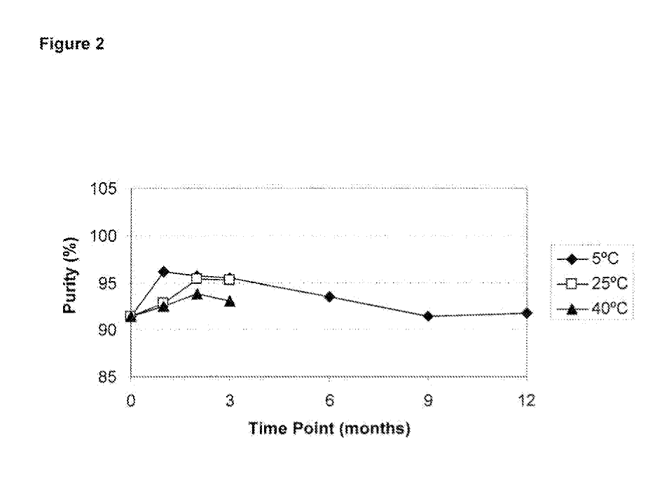

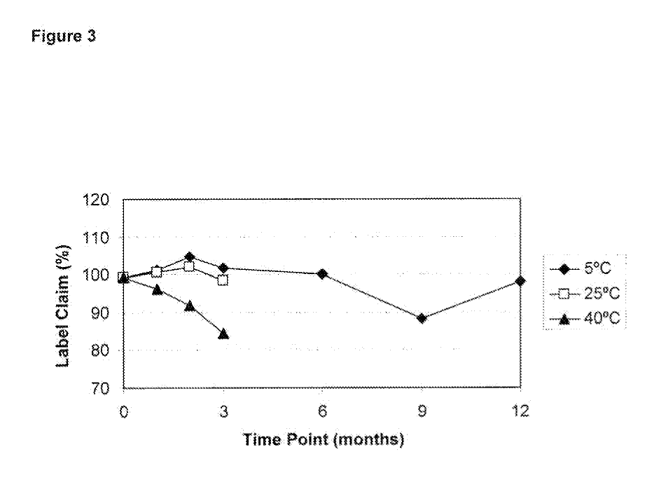

The stability of the lyophilized NY-ESO-1 peptide formulation was also assessed based on purity and percent label claim, as described in Table 2, under various conditions of up to twelve months at the storage condition of 5° C., and three months at the accelerated conditions of 25° C. and 40° C. As depicted in FIG. 2, the purity remained greater than 90% for all times and conditions tested. FIG. 3 depicts the percent label claim which remained greater than 85% for all time points at 5° C. and 25° C. At 40° C., the three-month sample was below 85%.

TABLE 3
3-month Stability Results for Formulation 1; Buffer A

| | | Cake | Suspension |
|---|---|---|---|
| HPLC | mg/vial | Appearance | Appearance |
| 5° C. Vial 1 | 0.503 | firm, white | Hazy |
| 5° C. Vial 2 | 0.510 | firm, white | Hazy |
| 5° C. Vial 3 | 0.511 | firm, white | Hazy |
| 5° C. Average | 0.508 | | |
| 25° C. Vial 1 | 0.495 | broken, white | Hazy |
| 25° C. Vial 2 | 0.486 | broken, white | Hazy |
| 25° C. Vial 3 | 0.496 | broken, white | Hazy |
| 25° C. Average | 0.492 | | |
| 40 C Vial 1 | 0.423 | broken, white | Hazy |
| 40° C. Vial 2 | 0.429 | firm, white | Hazy |
| 40° C. Vial 3 | 0.415 | broken, white | Hazy |
| 40° C. Average | 0.422 | | |

| Moisture | 25° C., wt % H₂O | 40° C., wt % H₂O |
|---|---|---|
| Vial 1 | 0.8 | 1.1 |
| Vial 2 | 0.9 | 1.2 |
| Average | 0.9 | 1.2 |

TABLE 4
3-month Stability Results for Formulation 2; Buffer B

| | | Cake | Suspension |
|---|---|---|---|
| HPLC | mg/vial | Appearance | Appearance |
| 5° C. Vial 1 | 0.512 | firm, white | Hazy |
| 5° C. Vial 2 | 0.511 | firm, white | Hazy |
| 5° C. Vial 3 | 0.502 | broken, white | Hazy |
| 5° C. Average | 0.508 | | |
| 25° C. Vial 1 | 0.486 | firm, white | Hazy |
| 25° C. Vial 2 | 0.494 | broken, white | Hazy |
| 25° C. Vial 3 | 0.491 | firm, white | Hazy |
| 25° C. Average | 0.490 | | |
| 40 C Vial 1 | 0.387 | broken, white | Hazy |
| 40° C. Vial 2 | 0.413 | broken, white | Hazy |
| 40° C. Vial 3 | 0.429 | broken, white | Hazy |
| 40° C. Average | 0.410 | | |

| Moisture | 25° C., wt % H₂O | 40° C., wt % H₂O |
|---|---|---|
| Vial 1 | 0.6 | 1.1 |
| Vial 2 | 0.7 | 1.2 |
| Average | 0.7 | 1.2 |

### Example 5

### Particle Size Distribution Suspended Formulations

Additionally, studies were conducted to evaluate the particle size distribution of the formulations after suspension in order to establish both process consistency and physical stability of the particle suspension (short-term and long-term). The particle size distribution evaluation served to determine if the lyophilized cakes formed a stable fine suspension after storage and disintegration. If the median (X₅₀) particle size did not increase significantly upon storage of the suspension, it indicated that there was no significant agglomeration, and therefore, the suspension was physically stable under the conditions tested.

This was accomplished by disintegrating each of the lyophilized cakes in 1 ml of disintegration buffer. The disintegration buffers for each formulation were also evaluated. As described in the stability protocol in Table 2 above, particle size distribution of the disintegrated lyophilized cake suspension was evaluated by laser light scattering using Sympatec's HELOS particle sizing instrument.

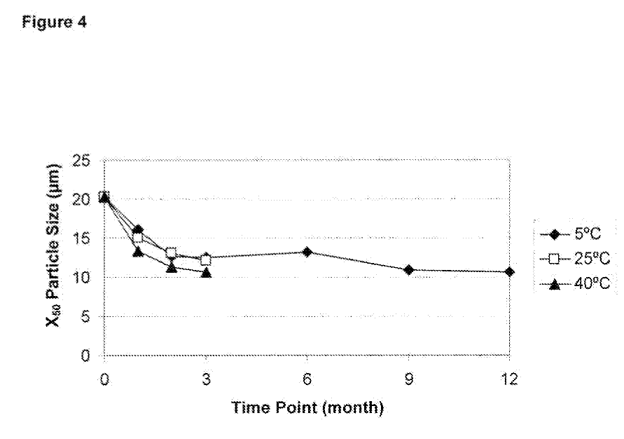

As shown in FIG. 4, the median (X₅₀) particle size declined from 20 μm over the first two months for all temperatures tested, and stabilized between 10 and 15 μm for the remainder of the time tested. The data indicates that the particles of NY-ESO-1 did not agglomerate during storage when prepared by the methods and processes disclosed herein.

### Example 6

### Particle Size Distribution at Six Hours Period Post-Disintegration

The particle size was also evaluated to assess whether the suspension created by the disintegration of the rake remained stable over a short period of time, for example, six hours. Lyophilized NY-ESO-1 peptide cakes obtained from Formulation 1, stored for three months at 5° C., 25° C. and 40° C., were disintegrated with 1 ml of sodium phosphate buffer containing 1% polysorbate 80. The resulting suspension contained the NY-ESO-1 peptide (1.0 mg/mL), mannitol (4%), polysorbate 80 (1.5%), and pH 8 phosphate buffer (50 mM). The X₉₀, particle size was evaluated immediately after disintegration and at six hours post-disintegration.

The data presented in Table 5 indicated that after three months of storage, the lyophilized NY-ESO-1 peptide cake from Formulation 1, upon disintegration in 1 ml of sodium phosphate buffer containing 1% polysorbate 80 produces a suspension that is stable at room temperature for at least six hours, as the size of the particles (20-30 microns) did not significantly change over this period of time tested. The storage temperature of the cake did not seem to affect the suspension stability.

TABLE 5
Particle size distribution of Formulation 1 immediately
after disintegration in Buffer and six hours later.

X₉₀ (μm)*

| Temperature | Time = 0 hr | Time = 6 hr |
|---|---|---|
| 5° C. | 24.07 | 23.35 |
| | 23.02 | 26.61 |
| 25° C. | 24.5 | 25.25 |
| | 24.75 | 23.60 |
| 40° C. | 23.89 | 24.91 |
| | 18.74 | 21.21 |

*X₉₀ indicates that 90% of particles are of a size below the value reported

Lyophilized cakes obtained from Formulation 2, stored for three months at 5° C., were also evaluated as described above for Formulation 1. Upon disintegration of Formulation 2 in 1 ml of sodium phosphate buffer containing 0.5% polysorbate 80, it was observed that Formulation 2 produced particles of various sizes, some of them exceeding 100 microns in size, while the freshly prepared formulation upon disintegration, did not contain particles of sizes larger than 20-30 microns. This suggests that the higher amount of polysorbate 80 in the cake may have led to aggregation during the three-month storage at this temperature.

The data indicates that Formulation 1 which contained a lower amount of polysorbate 80 (e.g., 0.5%) at the time of storage, compared to the Formulation 2 stored with a higher amount of polysorbate 80 (e.g., 1%), showed minimal change in particle size immediately after disintegration in buffer and six hours thereafter. Additionally, based on the evaluation of the cake appearance and suspension particle size of the formulations tested. Formulation 1 appeared to be a more stable formulation than Formulation 2 and is thus recommended for clinical applications.

### Example 7

### Analysis of the Potency of the Formulations

In addition to the parameters evaluated as described in the above examples, the potency of the suspension obtained from the lyophilized cakes of Formulation 1 stored at 25° C. was also examined. Lyophilized cakes stored at 25° C. for three months were removed from storage disintegrated in Buffer A and tested for potency as described in Table 2 above.

Table 6, shows an average binding of 81% of the NY-ESO-1₁₅₇₋₁₆₅ analogue S(Nva)LMWITQV (SEQ ID NO:3) to the MHC molecule HLA-A*0201. Additionally, the length of time the peptide remained bound to the MHC molecule was also determined using the half-life (t_{1/2},) assay described in Table 2 above. Peptides that do not quickly dissociate from the MHC molecule are generally more immunogenic. It is noted, in Table 6 that 50% of the NY-ESO-1 peptide analogue molecules remained bound to MHC molecules at an average t_{1/2} of 13 hours. Each of the assay control (NY-ESO-1 peptide analogue SEQ ID NO:3), bulk peptide stored at −80° C.) and Formulation 1 stored at room temperature for three months was analyzed in triplicate. The positive control showed a t_{1/2} of 10 and A0201 percent binding of 100%.

The data presented in Table 6 shows that there was no loss of binding affinity or avidity of the lyophilized formulation as compared to peptide prepared freshly from bulk.

TABLE 6
Potency assay

| NY-ESO-1₁₅₇₋₁₆₅ | | |
|---|---|---|
| (L158Nva, C165V) | Half-Life | A0201 |
| Peptide Formulation | (T_{1/2}) | (% Binding) |
| Assay Control | 14.1 | 80.4 |
| (bulk peptide ) | 13.8 | 79.3 |
| | 13.7 | 79.3 |
| Formulation 1 | 13.5 | 81.5 |
| (stored for 3 months) | 12.9 | 82.6 |
| | 12.56 | 80.4 |

### Example 8

### Intranodal Delivery of the Formulations

To determine the potential of the formulation process and the excipients used to cause a loss of immunogenicity, formulations containing the NY-ESO-1 peptide analogue (SEQ ID NO:3) were disintegrated in both Buffer A and B and the cellular immune responses measured for each formulation by interferon-gamma ELISPOT assay and chromium release assay as described in the Examples below.

Seven groups of female HEED-1 transgenic mice (n=8) were immunized with the NY-ESO-1 formulations plus polyI:C as adjuvant via bilateral inguinal lymph node injection on days 1, 4, 15 and 18 with 25 μg peptide/dose, (total dose=200 μg). Each of two groups of mice were immunized with NY-ESO-1 Formulation 1, Formulation 2 or Formulation 3 as shown in Table 7 and disintegrated in 1 ml of Buffer A or B as shown in Table 8. One group of mice was immunized with the peptide as a crude suspension in PBS buffer (control group). The mice were anesthetized using isoflurane and an incision approximately 0.5 cm in length was made in the inguinal fold to expose the inguinal lymph node. 25 μl (12.5 μg peptide) was injected directly into each of 2 contralateral inguinal lymph nodes using a 0.5-mL insulin syringe for a total dose of 25 μg/mouse. The wound was then closed with sterile 6-0 nylon skin sutures (PolyI:C was used in all samples as an adjuvant in determining an immune response in mice).

TABLE 7
Components of NY-ESO-1 Formulations

| Components | Formulation 1 | Formulation 2 | Formulation 3 |
|---|---|---|---|
| NY-ESO-1₁₅₇₋₁₆₅ | 0.5 mg/mL | 0.5 mg/mL | 0.75 mg/mL |
| (L158Nva, C165V) | | | |
| Mannitol | 4% | 4% | 4% |
| Polysorbate 80 | 0.5% | 1.0% | 0.5% |

TABLE 8
Excipient Effect on Immunogenicity - Experimental design

| | | | | | | Total |
|---|---|---|---|---|---|---|
| | Mice/ | | | Dose | Total | Dose |
| Group | group | Route¹ | Formulation^{3,4} | μg² | Cycle | (μg) |
| G1 | 8 | IN | NY-ESO-1 | 25 (0.833) | 2 | 200 |
| | | | Formulation 1: | | | |
| | | | 0.5 mg in | | | |
| | | | 1 ml Buffer A | | | |
| G2 | 8 | IN | NY-ESO-1 | 25 (0.833) | 2 | 200 |
| | | | Formulation 1: | | | |
| | | | 0.5 mg in | | | |
| | | | 1 ml Buffer B | | | |
| G3 | 8 | IN | NY-ESO-1 | 25 (0.833) | 2 | 200 |
| | | | Formulation 2: | | | |
| | | | 0.5 mg in | | | |
| | | | 1 ml Buffer A | | | |
| G4 | 8 | IN | NY-ESO-1 | 25 (0.833) | 2 | 200 |
| | | | Formulation 2: | | | |
| | | | 0.5 mg in | | | |
| | | | 1 ml Buffer B | | | |
| G5 | 8 | IN | NY-ESO-1 | 25 (0.833) | 2 | 200 |
| | | | Formulation 3: | | | |
| | | | 0.5 mg in | | | |
| | | | 1 ml Buffer A | | | |
| G6 | 8 | IN | NY-ESO-1 | 25 (0.833) | 2 | 200 |
| | | | Formulation 3: | | | |
| | | | 0.5 mg in | | | |
| | | | 1 ml Buffer B | | | |
| G7 | 8 | IN | NY-ESO-1 | 25 (0.833) | 2 | 200 |
| | | | Control: 0.5 mg | | | |
| | | | in 1XPBS | | | |

¹IN—Inguinal Intranodal Injection
²Peptide formulated at a concentration of 0.5 mg/mL
³Buffer A: 50 mM phosphate pH8 + 0.5% polysorbate 80; Buffer B: 50 mM phosphate pH 8
⁴0.5 mg polyl: C was used in all samples

### ELISPOT Analysis

The cellular immune response in immunized animals was measured using ELISPOT for IFN-γ. For quantification of IFN-γ producing cells, spleens were isolated on day 21 and single cell suspensions were prepared (n=7). Splenocytes (3×10⁵ or 1×10⁵ cells per well) from HHD-1 transgenic mice were incubated with 10 μg of NY-ESO-1₁₅₇₋₁₆₅ peptide (SEQ ID NO:3), in triplicate wells of a 96-well filter membrane plate (Multi-screen IP membrane 96-well plate, Millipore). Samples were incubated for 24 hours at 37° C. with 5% CO₂ and 100% humidity prior to development. Mouse IFN-γ coating antibody (IFN-γ antibody pair, Ucytech) was used as a coating reagent prior to incubation with splenocytes, followed by the biotinylated detection antibody.

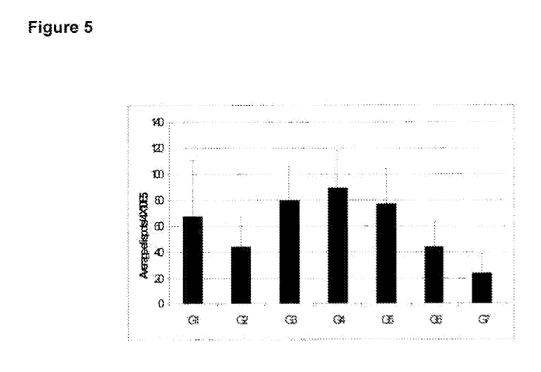

The IFN-γ ELISPOT results shown in FIG. 5 indicate that the formulated NY-ESO peptide (all 3 formations, in both buffers) appear to have somewhat increased ability to induce IFN-γ producing cells as compared to the NY-ESO-1 peptide as a crude suspension in PBS, presumably by increasing the bioavailability of the peptide once administered to the inguinal lymph node. There was no apparent correlation between polysorbate 80 concentration and cellular immune (IFN-γ) response.

### Example 9

### ⁵¹Chromium-Release Assay Measuring CTL Activity

The possible impairment of immunogenicity by the formulation process and the excipients used was also assessed by ⁵¹Chromium-release assay.

Splenocytes (5×10⁶ cells per well) from the immunized mice were plated in 24-well tissue culture plates and 1.5×10⁶ peptide-pulsed, γ-irradiated and LPS (lipopolysaccharide) blasted B cells were added to each well. Mouse recombinant IL-2 was also added at a concentration of 1 ng/ml. The cells were incubated for 6 days at 37° C. with 5% CO₂. After the ex vivo stimulation, CTLs were collected from the plates, washed, and plated into 96-well U-bottom micro-titer assay plates at concentrations of 10⁶, 3.3×10⁵, and 1.1×10⁵ cells/well in a total of 100 μL per well. To assess peptide specific lysis, T2 cells were labeled with ⁵¹Cr and pulsed with 20 μg/mL of NY-ESO₁₅₇₋₁₆₅ (L158Nva, C165V) analogue, at 37° C. for 1.5 hours. After the incubation, the cells were washed and resuspended. A quantity of ⁵¹Cr-labeled and peptide-pulsed T2 cells was added to each well. The cells were then incubated at 37° C. for 4 hours. After incubation, supernatants were harvested and the cytolytic activity was measured in triplicate samples using a gamma counter. The corrected percent lysis was calculated for each concentration of effector cells, using the mean cpm for each replicate of wells. Percent specific lysis was calculated using the following formula: Percent release=100×(Experimental release−spontaneous release)/(Maximum release−spontaneous release). Data are presented as follows: on the x-axis, the effector to target ratio is indicated; on the y-axis the corresponding percentage specific lysis is shown.

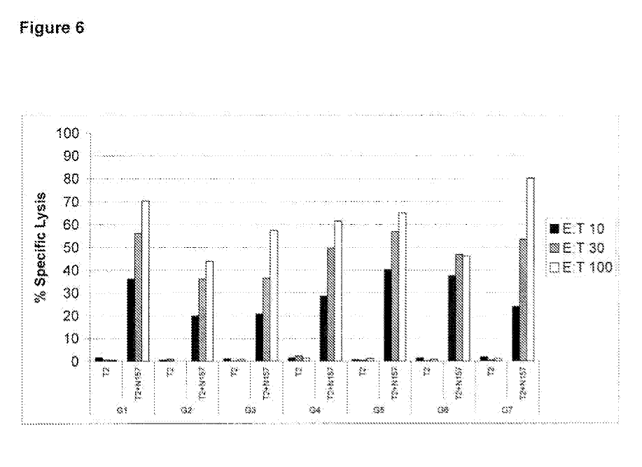

FIG. 6 shows ⁵¹Cr release assay data for CTL from each formulation group against T2 cells pulsed with NY-ESO-1₁₅₇₋₁₆₅ analogue peptide (SEQ ID NO:3); (T2+N157; FIG. 6) as targets. Specific lysis values were compared to un-pulsed T2 control cells (T2; FIG. 6). It was found that after in vitro re-stimulation, T cells isolated from all immunized groups specifically killed T2 cells pulsed with peptide. Comparable CTL responses to NY-ESO-1₁₅₇₋₁₆₅ analogue (SEQ ID NO:3) were induced in all groups, as assessed by ⁵¹Cr release cytotoxicity assays. These CTLs had no effect on T2 control cells without peptide. These results demonstrated that T2 target cell lysis by the CTLs isolated from the immunized mice is peptide specific.

Overall, the IFNγ ELISPOT results shown in FIG. 5 correlated well with the CRA data (FIG. 6) indicating that the immunogenicity of the NY-ESO analogue peptide (SEQ ID NO:3) administered as Formulation 1, 2 and 3, suspended in either Buffer A or B, was similar to that of the NY-ESO-1 analogue peptide (SEQ ID NO:3) administered as a crude suspension in PBS. There was no apparent correlation between polysorbate 80 concentration and immune response. These formulations did not impair the immunogenicity of the peptide.

Having described the disclosure in detail, it will be apparent that modifications, variations, and equivalent embodiments are possible without departing from the scope of the disclosure defined in the appended claims. Furthermore, it should be appreciated that all examples herein are provided as non-limiting examples.

In some embodiments, the terms “a” and “an” and “the” and similar referents used in the context of describing a particular embodiment of the disclosure (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Some embodiments of this disclosure are described herein, including the best mode known to the inventors for carrying out the disclosure. Variations on those embodiments will become apparent to those of ordinary skill in the art upon reading the foregoing description. It is contemplated that skilled artisans can employ such variations as appropriate, and the disclosure may be practiced otherwise than specifically described herein. Accordingly, many embodiments of this disclosure include all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in her entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the disclosure are illustrative of the principles of the present disclosure. Other modifications that can be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A method for preparing a lyophilized cake comprising a water-insoluble agent, wherein the lyophilized cake is capable of being disintegrated in a parenterally acceptable solvent to form an injectable formulation, the method comprising:
a) preparing a pre-lyophilization solution comprising: (i) one or more active agents, wherein at least one of the one or more active agents is water-insoluble; (ii) a parenterally unacceptable volatile lyophilizable solvent; and (iii) a water soluble bulking agent;
b) sterile filtering the pre-lyophilization solution thereby forming a sterile solution; and
c) lyophilizing the sterile solution to produce the lyophilized cake comprising the water-insoluble agent, wherein the lyophilized cake is capable of being disintegrated in the parenterally acceptable solvent to form the injectable formulation comprising a suspension of fine particles of the water-insoluble active agent.

2. The method of claim 1, wherein the pre-lyophilization solution further comprises a surfactant.

3. The method of claim 1, wherein preparing the pre-lyophilization solution comprises directly dissolving the at least one water-insoluble active agent and the water-soluble bulking agent in the parenterally unacceptable volatile lyophilizable solvent.

4. The method of claim 1, wherein preparing the pre-lyophilization solution comprises:
a) dissolving the at least one water-insoluble active agent in the parenterally unacceptable volatile lyophilizable solvent, thereby forming a first solution;
b) dissolving the water-soluble bulking agent, and optionally, a surfactant, in an aqueous solution thereby forming a second solution;
c) separately sterile filtering each of the first and second solutions to form a first sterile solution and a second sterile solution, respectively; and
d) adding the second sterile solution to the first sterile solution by controlled mixing to prevent aggregation or precipitation of the water-insoluble agent.

5. The method of claim 1, wherein the wherein the water-insoluble agent is hydrophobic.

6. The method of claim 1, wherein the water-insoluble agent is a small molecule, protein, or peptide.

7. (canceled)

8. The method of claim 1, wherein the water-insoluble agent is an immunogenic, therapeutic, or prophylactic molecule.

9. (canceled)

10. The method of claim 1, wherein the water-insoluble agent is a tumor antigen selected from the group consisting of NY-ESO-1, SSX-2, Melan-A, tyrosinase, PRAME, PSMA, and immunogenic fragments thereof.

11. (canceled)

12. The method of claim 10, wherein the immunogenic fragment comprises NY-ESO-1₁₅₇₋₁₆₅, or an analogue thereof.

13. The method of claim 12, wherein the NY-ESO-1₁₅₇₋₁₆₅ analogue is SNvaLMWITQV (SEQ ID NO:3).

14. The method of claim 1, wherein the parenterally unacceptable volatile lyophilizable solvent is an acid or base.

15. The method of claim 14, wherein the parenterally unacceptable volatile lyophilizable solvent is acetic acid or hydrochloric acid.

16. The method of claim 1, wherein the water-soluble bulking agent comprises mannitol.

17. The method of claim 1, further comprising a step for improving long-term stability against oxidative degradation of a hydrophobic agent having poor solubility in aqueous media, comprising storing the lyophilized cake under an inert gas.

18. A method for preparing an injectable formulation comprising a suspension of fine particles of a water-insoluble active agent, the method comprising:
obtaining a sterile lyophilized cake prepared according the method of claim 1; and
disintegrating the lyophilized cake in a parenterally acceptable solvent to form a liquid fine particle suspension for administration.

19. (canceled)

20. The method of claim 18, wherein the suspension is stable for about 360 minutes at room temperature.

21. (canceled)

22. The method of claim 18, wherein administration is directly to the lymphatic system.

23. (canceled)

24. The method of claim 18, wherein the lyophilized cake is disintegrated in water for injection, a sodium chloride solution, or a phosphate buffer.

25. A lyophilized cake comprising a water-insoluble agent, wherein the lyophilized cake is capable of being disintegrated in a parenterally acceptable solvent to form an injectable formulation comprising a suspension of fine particles of the water-insoluble active agent.

26. The lyophilized cake of claim 25, wherein the cake is prepared according to a method, wherein the method comprising:
a) preparing a pre-lyophilization solution comprising: (i) one or more active agents, wherein at least one of the one or more active agents is water-insoluble; (ii) a parenterally unacceptable volatile lyophilizable solvent; and (iii) a water soluble bulking agent;
b) sterile filtering the pre-lyophilization solution thereby forming a sterile solution; and
c) lyophilizing the sterile solution to produce the lyophilized cake comprising the water-insoluble agent, wherein the lyophilized cake is capable of being disintegrated in the parenterally acceptable solvent to form the injectable formulation comprising a suspension of fine particles of the water-insoluble active agent.

27. A kit comprising: i) a lyophilized cake comprising a water-insoluble agent, wherein the lyophilized cake is capable of being disintegrated in a parenterally acceptable solvent to form an injectable formulation comprising a suspension of fine particles of the water-insoluble active agent, ii) a parenterally acceptable solvent for preparing a fine particle suspension prior to administration, and iii) instructions for preparing the fine particle suspension.

28. The kit of claim 27, wherein the lyophilized cake is prepared according to a method, wherein the method comprising:
a) preparing a pre-lyophilization solution comprising: (i) one or more active agents, wherein at least one of the one or more active agents is water-insoluble; (ii) a parenterally unacceptable volatile lyophilizable solvent; and (iii) a water soluble bulking agent;
b) sterile filtering the pre-lyophilization solution thereby forming a sterile solution; and
c) lyophilizing the sterile solution to produce the lyophilized cake comprising the water-insoluble agent, wherein the lyophilized cake is capable of being disintegrated in the parenterally acceptable solvent to form the injectable formulation comprising a suspension of fine particles of the water-insoluble active agent.

29-32. (canceled)

33. A pharmaceutical composition comprising a lyophilized cake comprising one or more active agents, dispersed within the cake, wherein at least one of the one or more active agents is water-insoluble, and wherein upon disintegration of the cake with a parenterally acceptable solvent, a syringeable suspension of fine particles of the water-insoluble active agent is obtained.
